(19) United States
(12) Patent Application Publication
(10) Pub. No.: US 2021/0171613 A1
(43) Pub. Date: Jun. 10, 2021

(54) CLOSTRIDIUM DIFFICILE-SPECIFIC ANTIBODIES AND USES THEREOF

(71) Applicant: National Research Council of Canada, Ottawa (CA)

(72) Inventors: Traian SULEA, Kirkland (CA); Gregory HUSSACK, Ottawa (CA); Jamshid TANHA, Orleans (CA); Enrico O. PURISIMA, Pierrefonds (CA)

(73) Assignee: National Research Council of Canada, Ottawa, ON (CA)

(21) Appl. No.: 16/630,714

(22) PCT Filed: Jul. 9, 2018
(86) PCT No.: PCT/IB2018/055047

§ 371 Date: Jan. 13, 2020

Related U.S. Application Data
(60) Provisional application No. 62/531,009, filed on Jul. 11, 2017.

(51) Int. Cl. C07K 16/12; G01N 33/569

(52) U.S. Cl. CPC C07K 16/1282; G01N 33/56911; C07K 2317/22; C07K 2317/565; G01N 2333/33; C07K 2317/76; C07K 2317/92; C07K 2317/94; C07K 2317/569

(57) ABSTRACT

The present invention is directed to Clostridium difficile toxin-specific antibodies, compositions, and uses thereof. The anti-toxin antibodies may be specific for TcdA. The invention also includes methods of treating a Clostridium difficile infection, methods of capturing Clostridium difficile toxins, and methods of detecting Clostridium difficile toxins.

### CROSS-REFERENCE TO RELATED APPLICATIONS

This application is a national phase entry of International Patent Application No. PCT/162018/055047 filed Jul. 9, 2018, and claims the benefit of U.S. Provisional Patent Application Ser. No. 62/531,009 filed Jul. 11, 2017, the entire contents of both are herein incorporated by reference.

### FIELD OF THE INVENTION

The present invention relates to Clostridium difficile-specific antibodies and uses thereof. More specifically, the present invention relates to Clostridium difficile toxin-specific antibodies and uses thereof.

### BACKGROUND OF THE INVENTION

Clostridium difficile is a Gram-positive, anaerobic, endospore-forming gastrointestinal pathogen responsible for C. difficile-associated disease (CDAD) in humans and animals with symptoms ranging in severity from mild cases of antibiotic-associated diarrhea to fatal pseudomembranous colitis (Rupnik et al, 2009; Leffler and Lamont, 2009; Songer, 2004; Kelly et al, 1994). Each year in North America, 1-3% of hospitalized patients receiving antibiotics become infected with C. difficile, leading to thousands of deaths and over $1 billion in associated costs to the health-care system (Wilkins and Lyerly, 2003; Kyne et al, 2002; Kelly et al, 1994). C. difficile produces two primary virulence factors, toxin A (TcdA) and toxin B (TcdB), which are large (308 kDa and 269 kDa, respectively), single-subunit exotoxins composed of a catalytic, a translocation and a cell-receptor binding domain (RBD) (Jank and Aktories, 2008; Jank et al, 2007). Recently it was suggested TcdB is solely responsible for C. difficile virulence (Lyras et al, 2009), although earlier studies have shown both anti-TcdA and anti-TcdB monoclonal antibodies (mAbs) were required for full protection of hamsters from CDAD (Babcock et al, 2006; Kink and Williams, 1998) and anti-TcdA mAbs were required for protection in mice (Corthier et al, 1991).

The current approach for treating most CDAD infections involves administration of antibiotics, most commonly metronidazole or vancomycin (Leffler and Lamont, 2009). Antibiotic treatment places selection pressure on the organism, can lead to antibiotic resistance, and suppresses or eliminates beneficial commensal microbes. However, there are several other emerging challenges warranting the development of novel therapeutics. First, there is no acute CDAD treatment targeting TcdA/B. These toxins are responsible for loss of epithelial barrier function in the colon by disrupting tight junctions and increasing membrane permeability, causing diarrhea and promoting severe inflammation (Rupnik et al, 2009; Jank and Aktories, 2008). Second, hypervirulent strains of C. difficile, such as the NAP1/027 isolate, over-express TcdA and TcdB (Warny et al, 2005) and have been associated with increased mortality rates and disease severity (O'Connor et al, 2009; Pépin et al, 2005). Third, an estimated 20-25% of patients suffering from CDAD experience symptomatic relapse after the initial infection is cleared, with 45% of these patients prone to subsequent relapses (Johnson, 2009). Taken together, there is a need for non-antibiotic based reagents which target and inhibit TcdA and TcdB for CDAD therapy.

Individuals who are asymptomatic C. difficile carriers and patients who experience mild cases of CDAD tend to possess high anti-toxin A titers (Kyne et al, 2001; Kyne et al, 2000; Warny et al, 1994; Viscidi et al, 1983). Conversely, patients susceptible to relapsing C. difficile infection have low anti-TcdA immunoglobulin titers, specifically IgM, IgG2 and IgG3 isotypes (Katchar et al, 2007; Kyne et al, 2001). TcdA-neutralizing secretory IgA antibodies are also thought to play a role in regulating CDAD severity (Johal et al 2004; Kelly et al 1992). Therefore, the introduction of anti-toxin antibodies to patients suffering from severe C. difficile infection may be a therapeutically useful approach.

A limited number of animal and human studies have illustrated the effectiveness of anti-toxin Abs for treatment of CDAD. Babcock et al (2006) intravenously administered anti-TcdA and anti-TcdB mAbs to hamsters and found a significant reduction in hamster mortality in prophylactic, primary disease and relapse models when both anti-toxin mAbs were administered. A recently completed clinical trial involving these two humanized mAbs appears promising (Lowy et al, 2010). In another study, intravenous administration of anti-TcdA mAbs raised against the RBD followed by oral challenge with C. difficile resulted in protection of mice (Corthier et al, 1991). Elsewhere, a toxoid vaccine given by the intraperitoneal route to hamsters conferred protection against oral C. difficile challenge (Giannasca et al, 1999) and mice vaccinated with DNA encoding the TcdA RBD resulted in full protection from oral TcdA challenge (Gardiner et al, 2009). In humans, a number of uncontrolled studies have reported intravenous immunoglobulin (IVIG) therapy to be successful for the treatment of severe CDAD (Juang et al, 2007; Hassoun and Ibrahim, 2007; McPherson et al, 2006; Wilcox, 2004; Salcedo et al, 1997; Leung et al, 1991). IVIG involves administration of high concentrations (150-400 mg/kg) of human immunoglobulins from healthy donors which are thought to contain neutralizing anti-toxin antibodies as an estimated 60% of healthy adults have detectable TcdA- and TcdB-specific serum IgG antibodies (Viscidi et al, 1983).

Given that C. difficile toxins rely on attachment to epithelial cells for entry (Jank and Aktories, 2008; Jank et al, 2007), neutralizing the toxins within the lower gastrointestinal tract with antibodies may block the first step in CDAD pathogenesis. In animals, orally administered bovine immunoglobulin concentrate (BIC) containing TcdA and TcdB neutralizing IgGs were able to prevent hamster mortality when used as a propholyactic (Lyerly et al, 1991) and protected rats from the enterotoxic effects of TcdA in vivo (Kelly et al, 1996). Chicken IgY antibodies specific for toxin RBDs were shown to reduce hamster mortality when administered orally to infected animals (Kink and Williams, 1998). In humans, there have been limited reports on CDAD therapy with orally delivered Abs. Tjellström et al (1993) reported the successful treatment of a 3½ year old boy suffering from severe CDAD with IgA antibody orally. Warny et al (1999) and Kelly et al (1997) examined the passage of anti-toxin bovine IgG through the human gastrointestinal tract and found a significant reduction in IgG activity, likely due to proteolytic degradation within the upper gastrointestinal tract. The limited success of both oral and systemic anti-toxin immunotherapy in clinical settings has likely been hampered by the high immunoglobulin dose requirements (150-400 mg/kg), the associated costs of these doses, and a lack of published clinical data showing the effectiveness of these treatments.

Despite such advances, there remains a need in the art for a safe and effective therapeutic for treating C. difficile-associated disease as well as for sensitive and effective reagents for the detection of toxins A and B, the factors responsible for C. difficile-associated disease.

### SUMMARY OF THE INVENTION

The present invention relates to Clostridium difficile-specific antibodies and uses thereof. More specifically, the present invention relates to Clostridium difficile toxin-specific antibodies and uses thereof.

The present invention provides an isolated, purified or recombinant antibody or fragment comprising a sequence of CDR1 of ERTFSRYP (SEQ ID NO:1); CDR2 of ISSX₁GX₂SX₃ (SEQ ID NO:2); and CDR3 of AVNSX₄RX₅RLQDPX₆EYDY (SEQ ID NO: 3); wherein: X₁ is T or R; X₂ is T, R or I; X₃ is T or K; X₄ is Q or K; X₅ is T, R, K, M or W; and X₆ is N or R; and combinations thereof; and with the proviso that when CDR2 is ISSTGTST (SEQ ID NO:4) then CDR3 is not AVNSQRTRLQDPNEYDY (SEQ ID NO: 5), and vice-versa (i.e. CDR2 is not ISSTGTST (SEQ ID NO:4) when CDR3 is AVNSQRTRLQDPNEYDY (SEQ ID NO: 5); and wherein the antibody or fragment thereof is specific to TcdA. The antibody or fragment binds to TcdA with greater affinity than an antibody or fragment comprising CDR1 of SEQ ID NO:1, CDR2 of SEQ ID NO:4, and CDR3 of SEQ ID NO:5.

The isolated, purified or recombinant antibody or fragment may be selected from the group consisting of: an isolated, purified or recombinant antibody or fragment thereof comprising a sequence of CDR1 of SEQ ID NO:1; CDR2 of SEQ ID NO:6; and CDR3 of SEQ ID NO: 5; an isolated, purified or recombinant antibody or fragment thereof comprising a sequence of CDR1 of SEQ ID NO:1; CDR2 of SEQ ID NO:7; and CDR3 of SEQ ID NO: 5; an isolated, purified or recombinant antibody or fragment thereof comprising a sequence of CDR1 of SEQ ID NO:1; CDR2 of SEQ ID NO:8; and CDR3 of SEQ ID NO: 5; an isolated, purified or recombinant antibody or fragment thereof comprising a sequence of CDR1 of SEQ ID NO:1; CDR2 of SEQ ID NO:9; and CDR3 of SEQ ID NO: 5; an isolated, purified or recombinant antibody or fragment thereof comprising a sequence of CDR1 of SEQ ID NO:1; CDR2 of SEQ ID NO:4; and CDR3 of SEQ ID NO: 10; an isolated, purified or recombinant antibody or fragment thereof comprising a sequence of CDR1 of SEQ ID NO:1; CDR2 of SEQ ID NO:4; and CDR3 of SEQ ID NO: 11; an isolated, purified or recombinant antibody or fragment thereof comprising a sequence of CDR1 of SEQ ID NO:1; CDR2 of SEQ ID NO:4; and CDR3 of SEQ ID NO: 12; an isolated, purified or recombinant antibody or fragment thereof comprising a sequence of CDR1 of SEQ ID NO:1; CDR2 of SEQ ID NO:4; and CDR3 of SEQ ID NO: 13; an isolated, purified or recombinant antibody or fragment thereof comprising a sequence of CDR1 of SEQ ID NO:1; CDR2 of SEQ ID NO:4; and CDR3 of SEQ ID NO: 14; an isolated, purified or recombinant antibody or fragment thereof comprising a sequence of CDR1 of SEQ ID NO:1; CDR2 of SEQ ID NO:4; and CDR3 of SEQ ID NO: 15; an isolated, purified or recombinant antibody or fragment thereof comprising a sequence of CDR1 of SEQ ID NO:1; CDR2 of SEQ ID NO:6; and CDR3 of SEQ ID NO: 10; an isolated, purified or recombinant antibody or fragment thereof comprising a sequence of CDR1 of SEQ ID NO:1; CDR2 of SEQ ID NO:6; and CDR3 of SEQ ID NO: 11; an isolated, purified or recombinant antibody or fragment thereof comprising a sequence of CDR1 of SEQ ID NO:1; CDR2 of SEQ ID NO:6; and CDR3 of SEQ ID NO: 12; an isolated, purified or recombinant antibody or fragment thereof comprising a sequence of CDR1 of SEQ ID NO:1; CDR2 of SEQ ID NO:6; and CDR3 of SEQ ID NO: 13; an isolated, purified or recombinant antibody or fragment thereof comprising a sequence of CDR1 of SEQ ID NO:1; CDR2 of SEQ ID NO:4; and CDR3 of SEQ ID NO: 16; an isolated, purified or recombinant antibody or fragment thereof comprising a sequence of CDR1 of SEQ ID NO:1; CDR2 of SEQ ID NO:4; and CDR3 of SEQ ID NO: 17; an isolated, purified or recombinant antibody or fragment thereof comprising a sequence of CDR1 of SEQ ID NO:1; CDR2 of SEQ ID NO:4; and CDR3 of SEQ ID NO: 18; an isolated, purified or recombinant antibody or fragment thereof comprising a sequence of CDR1 of SEQ ID NO:1; CDR2 of SEQ ID NO:6; and CDR3 of SEQ ID NO: 16; an isolated, purified or recombinant antibody or fragment thereof comprising a sequence of CDR1 of SEQ ID NO:1; CDR2 of SEQ ID NO:6; and CDR3 of SEQ ID NO: 17; an isolated, purified or recombinant antibody or fragment thereof comprising a sequence of CDR1 of SEQ ID NO:1; CDR2 of SEQ ID NO:6; and CDR3 of SEQ ID NO: 18.

The present invention also provides an isolated, purified or recombinant antibody or fragment that may be selected from the group consisting of:

QVKLEESGGGLVQAGGSLRLSCAASERTFSRYPVAWFRQAPGAEREFVAVISSX₁GX₂SX₃YYADSVKGRFTISRDNAKVTVYLQMNNLKREDTAVYFCAVNSX₄RX₅RLQDPX₆EYDYWGQ GTQVTVSS (SEQ ID NO: 19); wherein: X₁ is T or R; X₂ is T, R or I; X₃ is T or K; X₄ is Q or K; X₅ is T, R, K, M or W; and X₆ is N or R; and combinations thereof; and excluding SEQ ID NO: 20.

Furthermore, the present invention provides an isolated, purified or recombinant antibody or fragment that may be selected from the group consisting of:

(SEQ ID NO: 21)
QVKLEESGGGLVQAGGSLRLSCAASERTFSRYPVAWFRQAPGAEREFVAV
ISSRGTSTYYADSVKGRFTISRDNAKVTVYLQMNNLKREDTAVYFCAVNS
QRTRLQDPNEYDYWGQGTQVTVSS;
(SEQ ID NO: 22)
QVKLEESGGGLVQAGGSLRLSCAASERTFSRYPVAWFRQAPGAEREFVAV
ISSTGRSTYYADSVKGRFTISRDNAKVTVYLQMNNLKREDTAVYFCAVNS
QRTRLQDPNEYDYWGQGTQVTVSS;
(SEQ ID NO: 23)
QVKLEESGGGLVQAGGSLRLSCAASERTFSRYPVAWFRQAPGAEREFVAV
ISSTGISTYYADSVKGRFTISRDNAKVTVYLQMNNLKREDTAVYFCAVNS
QRTRLQDPNEYDYWGQGTQVTVSS;
(SEQ ID NO: 24)
QVKLEESGGGLVQAGGSLRLSCAASERTFSRYPVAWFRQAPGAEREFVAV
ISSTGKSTYYADSVKGRFTISRDNAKVTVYLQMNNLKREDTAVYFCAVNS
QRTRLQDPNEYDYWGQGTQVTVSS;
(SEQ ID NO: 25)
QVKLEESGGGLVQAGGSLRLSCAASERTFSRYPVAWFRQAPGAEREFVAV
ISSTGTSTYYADSVKGRFTISRDNAKVTVYLQMNNLKREDTAVYFCAVNS
KRTRLQDPNEYDYWGQGTQVTVSS;
(SEQ ID NO: 26)
QVKLEESGGGLVQAGGSLRLSCAASERTFSRYPVAWFRQAPGAEREFVAV
ISSTGTSTYYADSVKGRFTISRDNAKVTVYLQMNNLKREDTAVYFCAVNS
QRRRLQDPNEYDYWGQGTQVTVSS;
(SEQ ID NO: 27)
QVKLEESGGGLVQAGGSLRLSCAASERTFSRYPVAWFRQAPGAEREFVAV
ISSTGTSTYYADSVKGRFTISRDNAKVTVYLQMNNLKREDTAVYFCAVNS
QRKRLQDPNEYDYWGQGTQVTVSS;
(SEQ ID NO: 28)
QVKLEESGGGLVQAGGSLRLSCAASERTFSRYPVAWFRQAPGAEREFVAV
ISSTGTSTYYADSVKGRFTISRDNAKVTVYLQMNNLKREDTAVYFCAVNS
QRMRLQDPNEYDYWGQGTQVTVSS;
(SEQ ID NO: 29)
QVKLEESGGGLVQAGGSLRLSCAASERTFSRYPVAWFRQAPGAEREFVAV
ISSTGTSTYYADSVKGRFTISRDNAKVTVYLQMNNLKREDTAVYFCAVNS
QRWRLQDPNEYDYWGQGTQVTVSS;
(SEQ ID NO: 30)
QVKLEESGGGLVQAGGSLRLSCAASERTFSRYPVAWFRQAPGAEREFVAV
ISSTGTSTYYADSVKGRFTISRDNAKVTVYLQMNNLKREDTAVYFCAVNS
QRTRLQDPREYDYWGQGTQVTVSS;
(SEQ ID NO: 31)
QVKLEESGGGLVQAGGSLRLSCAASERTFSRYPVAWFRQAPGAEREFVAV
ISSTGRSTYYADSVKGRFTISRDNAKVTVYLQMNNLKREDTAVYFCAVNS
KRTRLQDPNEYDYWGQGTQVTVSS;
(SEQ ID NO: 32)
QVKLEESGGGLVQAGGSLRLSCAASERTFSRYPVAWFRQAPGAEREFVAV
ISSTGRSTYYADSVKGRFTISRDNAKVTVYLQMNNLKREDTAVYFCAVNS
QRRRLQDPNEYDYWGQGTQVTVSS;
(SEQ ID NO: 33)
QVKLEESGGGLVQAGGSLRLSCAASERTFSRYPVAWFRQAPGAEREFVAV
ISSTGRSTYYADSVKGRFTISRDNAKVTVYLQMNNLKREDTAVYFCAVNS
QRKRLQDPNEYDYWGQGTQVTVSS;
(SEQ ID NO: 34)
QVKLEESGGGLVQAGGSLRLSCAASERTFSRYPVAWFRQAPGAEREFVAV
ISSTGRSTYYADSVKGRFTISRDNAKVTVYLQMNNLKREDTAVYFCAVNS
QRMRLQDPNEYDYWGQGTQVTVSS;
(SEQ ID NO: 35)
QVKLEESGGGLVQAGGSLRLSCAASERTFSRYPVAWFRQAPGAEREFVAV
ISSTGTSTYYADSVKGRFTISRDNAKVTVYLQMNNLKREDTAVYFCAVNS
KRRRLQDPNEYDYWGQGTQVTVSS;
(SEQ ID NO: 36)
QVKLEESGGGLVQAGGSLRLSCAASERTFSRYPVAWFRQAPGAEREFVAV
ISSTGTSTYYADSVKGRFTISRDNAKVTVYLQMNNLKREDTAVYFCAVNS
KRKRLQDPNEYDYWGQGTQVTVSS;
(SEQ ID NO: 37)
QVKLEESGGGLVQAGGSLRLSCAASERTFSRYPVAWFRQAPGAEREFVAV
ISSTGTSTYYADSVKGRFTISRDNAKVTVYLQMNNLKREDTAVYFCAVNS
KRMRLQDPNEYDYWGQGTQVTVSS;
(SEQ ID NO: 38)
QVKLEESGGGLVQAGGSLRLSCAASERTFSRYPVAWFRQAPGAEREFVAV
ISSTGRSTYYADSVKGRFTISRDNAKVTVYLQMNNLKREDTAVYFCAVNS
KRRRLQDPNEYDYWGQGTQVTVSS;
(SEQ ID NO: 39)
QVKLEESGGGLVQAGGSLRLSCAASERTFSRYPVAWFRQAPGAEREFVAV
ISSTGRSTYYADSVKGRFTISRDNAKVTVYLQMNNLKREDTAVYFCAVNS
KRKRLQDPNEYDYWGQGTQVTVSS;
(SEQ ID NO: 40)
QVKLEESGGGLVQAGGSLRLSCAASERTFSRYPVAWFRQAPGAEREFVAV
ISSTGRSTYYADSVKGRFTISRDNAKVTVYLQMNNLKREDTAVYFCAVNS
KRMRLQDPNEYDYWGQGTQVTVSS; or a sequence
substantially identical thereto.

The isolated, purified or recombinant antibodies or fragments provided by the present invention neutralize the cytotoxicity of TcdA with an EC50 in the nanomolar concentration range. In certain embodiments, the isolated, purified or recombinant antibody or fragment can be selected from the group consisting of SEQ ID NO: 32 and SEQ ID NO: 38, which neutralizes the cytotoxicity of TcdA with an EC50 below the concentration of 100 nM and preferably with an EC50 of 20 nM or lower.

The isolated or purified antibody or fragment thereof as described above may be a single-domain antibody (sdAb); the sdAb may be of camelid origin.

The isolated or purified antibody or fragment thereof of as described herein may be in a multivalent display format.

The isolated or purified antibody or fragment thereof as described herein may be immobilized onto a surface.

The isolated or purified antibody or fragment thereof of the present invention may be linked to a cargo molecule; the cargo molecule may be a detectable agent, a therapeutic, a drug, a peptide, a protease, an enzyme, a carbohydrate moiety, or a cytotoxic agent; one or more liposomes loaded with a detectable agent, a therapeutic, a drug, a peptide, an enzyme, or a cytotoxic agent; or one or more nanoparticle, nanowire, nanotube, or quantum dots.

The present invention further encompasses a nucleic acid molecule encoding the isolated or purified antibody or fragment thereof as described above. The present invention also includes a vector comprising the nucleic acid molecule just described.

Also provided is a composition comprising one or more than one isolated or purified antibody or fragment thereof of the present invention and a pharmaceutically-acceptable carrier, diluent, or excipient.

The present invention further provides a method of treating a Clostridium difficile infection, comprising administering the isolated or purified antibody or fragment thereof of the present invention or the composition of described above to a subject in need thereof.

In another aspect, there is provided a method of capturing Clostridium difficile toxins, comprising contacting a sample with one or more than one isolated or purified antibody or fragment thereof of the present invention immobilized onto a surface, and allowing the toxin(s) to bind to the isolated or purified antibody or fragment thereof. The method just described may further comprise identifying the toxin by mass spectrometric methods and/or eluting the bound toxin.

The present invention additionally provides a method of detecting Clostridium difficile toxins, comprising contacting a sample with one or more than one isolated or purified antibody or fragment thereof linked to a cargo molecule, and detecting the bound antibody or fragment thereof using a suitable imaging or detection technology. The cargo molecule may be a detectable agent.

The present invention provides engineered single-domain antibodies (V_{H}Hs) capable of binding, detecting, capturing, and/or neutralizing C. difficile TcdA. Without wishing to be bound by theory, V_{H}Hs targeting the toxin's receptor binding domain (RBD) may block the toxin-receptor interaction, thereby preventing toxin entry into the host cell; a critical initial step in the TcdA mechanism of action (Jank and Aktories, 2008). To do so, a llama V_{H}H capable of binding to recombinant RBD fragments was optimized for enhanced binding affinity to TcdA using the ADAPT (Assisted Design of Antibody and Protein therapeutics) platform for affinity maturation (Vivcharuk et al, 2017)). The engineered V_{H}Hs were then characterized for their ability to bind native toxins and recombinant RBD fragments and the nature and optimized interaction with toxin epitopes. In addition, the ability of V_{H}Hs to neutralize toxins in an in vitro cell cytotoxicity assay was assessed.

Several TcdA-specific V_{H}Hs capable of neutralizing TcdA in vitro through high-affinity interactions with TcdA-RBD were found. V_{H}Hs are extremely stable antigen-binding domains that are expressed at high-yields in recombinant organisms and are capable of neutralizing infectious disease-related targets (Wesolowski et al, 2009). With respect to CDAD therapy, V_{H}Hs could be administered systemically to target TcdA as they share high sequence homology with human V_{H} domains, thus are well-tolerated in humans (Vu et al, 1997; www.Ablynx.com). Enhanced toxin neutralizing efficacy may be obtained by increasing their blood circulation half lives, size and avidity using various techniques, including chimeric formats of anti-TcdA V_{H}Hs linked to an Fc domain, generation of bi- or tri-specific antibody fusions with two or three anti-TcdA V_{H}Hs recognizing unique epitopes, PEGylation, fusion to serum albumin, or fusion to serum albumin-specific antibody fragments. By targeting C. difficile virulence factors such as TcdA/B, selection pressure is removed from the organism, decreasing the chance of antibiotic resistance. A mutation in the RBD, which is conserved among C. difficile isolates including hypervirulent 027 ribotype strains, is unlikely to benefit the organism and in the event it does occur, the toxin may lose its ability to enter host cells. As such, anti-TcdA V_{H}Hs are logical agents to explore for CDAD therapy.

Additional aspects and advantages of the present invention will be apparent in view of the following description. The detailed description and examples, while indicating preferred embodiments of the invention, are given by way of illustration only, as various changes and modifications within the scope of the invention will become apparent to those skilled in the art in light of the teachings of this invention.

### BRIEF DESCRIPTION OF THE DRAWINGS

These and other features of the invention will now be described by way of example, with reference to the appended drawings, wherein:

FIG. 1. Sequence of the parental anti-TcdA V_{H}H A26.8 (WO/2012/055030) and its sequential numbering adopted herein. CDR loop delineated according to the IMGT definition are shaded. Positions mutated leading to increase in vitro TcdA binding affinity are indicated by vertical arrows and labeled.

FIG. 2. Round 1 of ADAPT leading to validated affinity-improved single-point mutants of a C. difficile TcdA-binding V_{H}H. (A) Proximity of virtually scanned CDR residues to the TcdA epitope. (B) 3-D locations of positions where mutations were selected for experimental testing. (C) Single-point mutations found to lead to antigen binding affinity improvements of over 2-fold relative to parental V_{H}H. The antigen is shown as black/gray ribbon in a translucent molecular surface. The V_{H}H is rendered as a ribbon/Cα-trace, with CDR side chains shown as ball-and-stick/stick models. Models of mutated side-chains are shown in (C) as sticks.

FIG. 3. Ratios of wild-type to mutant K_{D}s (K_{D}^{WT}/K_{D}) and differences in binding free energies (ΔΔG, kcal/mol) relative to the parent V_{H}H during two rounds of mutations. Round 1 mutants that carried forward to Round 2 are highlighted in bold.

FIG. 4. Additivity of contributions of mutations to binding affinity. Scatter plot of experimentally measured relative binding affinities of double and triple mutants versus the sum of experimentally measured relative binding affinities of the component single mutants. The dashed line is the linear regression line for the entire data set, while the solid diagonal line indicates full additivity. Square symbols highlight mutants incorporating simultaneous substitutions at adjacent positions 101 and 103 with positively-charged amino acids.

FIG. 5. Representative SPR sensorgram binding profiles. Interaction of (A) the parent A26.8 V_{H}H, (B) the lead double mutant T56R,T103R, and (C) the lead triple mutant T56R,Q101K,T103R to immobilized full-length TcdA. The black lines represent raw data and the gray lines are global fits to a 1:1 bimolecular interaction model.

FIG. 6. Overview of thermal stabilities expressed as melting temperatures (Tₘs) measured by DSF for the A26.8 V_{H}H variants analyzed in this study. Dashed line represents the Tₘ of the wild-type A26.8 V_{H}H. Single mutants carried forward to the second round of ADAPT are highlighted in hashed bars.

FIG. 7. In vitro TcdA neutralization assay with affinity-matured V_{H}Hs. Vero cells were incubated with TcdA and increasing concentrations of V_{H}Hs. Cell proliferation was monitored spectrophotometrically relative to untreated controls and cells receiving TcdA only. Plotted are mean data from four independent assays.

FIG. 8. Molecular models of optimized TcdA-V_{H}H interactions. (A) and (B) represent different views showing interactions for mutations at positions 56, 101 and 103 of the V_{H}H. Panel (B) additionally includes an overlay of three different mutations at position 103. The TcdA is rendered in pale gray ribbon and the V_{H}H in dark gray ribbon. Mutated residues are rendered as ball-and-stick, other selected residues as sticks. H-bonds are indicated by black dashed lines.

### DETAILED DESCRIPTION OF THE INVENTION

The present invention relates to Clostridium difficile-specific antibodies and uses thereof. More specifically, the present invention relates to Clostridium difficile toxin-specific antibodies and uses thereof.

The present invention provides engineered single-domain antibodies (V_{H}Hs) capable of binding and neutralizing C. difficile TcdA. Without wishing to be bound by theory, V_{H}Hs targeting the toxin's receptor binding domain (RBD) may block the toxin-receptor interaction, thereby preventing toxin entry into the host cell; this represents a critical initial step in the TcdA mechanism of action (Jank and Aktories, 2008).

Thus, the present invention provides an isolated, purified or recombinant antibody or fragment comprising a sequence of CDR1 of ERTFSRYP (SEQ ID NO:1); CDR2 of ISSX₁GX₂SX₃ (SEQ ID NO:2); and CDR3 of AVNSX₄RX₅RLQDPX₆EYDY (SEQ ID NO: 3); wherein: X₁ is T or R; X₂ is T, R or I; X₃ is T or K; X₄ is Q or K; X₅ is T, R, K, M or W; and X₆ is N or R; and combinations thereof; and with the proviso that both CDR2 and CDR3 cannot be the corresponding CDR loops of the parental A26.8 V_{H}H (i.e., SEQ ID NO:4 and SEQ ID NO:5, respectively) at the same time; that is to say that CDR2 is not ISSTGTST (SEQ ID NO:4) when CDR3 is AVNSQRTRLQDPNEYDY (SEQ ID NO: 5), and when CDR2 is ISSTGTST (SEQ ID NO:4), CDR3 is not AVNSQRTRLQDPNEYDY (SEQ ID NO: 5), and vice-versa; wherein the antibody or fragment thereof is specific to TcdA. The antibody or fragment binds to TcdA with greater affinity than the single-domain antibody comprising CDR1 of SEQ ID NO:1, CDR2 of SEQ ID NO:4, and CDR3 of SEQ ID NO:5. The isolated or purified antibody or fragment thereof as just described may comprise a sequence of CDR1 of SEQ ID NO:1; CDR2 of SEQ ID NO:6; and CDR3 of SEQ ID NO: 5; an isolated, purified or recombinant antibody or fragment thereof comprising a sequence of CDR1 of SEQ ID NO:1; CDR2 of SEQ ID NO:7; and CDR3 of SEQ ID NO: 5; an isolated, purified or recombinant antibody or fragment thereof comprising a sequence of CDR1 of SEQ ID NO:1; CDR2 of SEQ ID NO:8; and CDR3 of SEQ ID NO: 5; an isolated, purified or recombinant antibody or fragment thereof comprising a sequence of CDR1 of SEQ ID NO:1; CDR2 of SEQ ID NO:9; and CDR3 of SEQ ID NO: 5; an isolated, purified or recombinant antibody or fragment thereof comprising a sequence of CDR1 of SEQ ID NO:1; CDR2 of SEQ ID NO:4; and CDR3 of SEQ ID NO: 10; an isolated, purified or recombinant antibody or fragment thereof comprising a sequence of CDR1 of SEQ ID NO:1; CDR2 of SEQ ID NO:4; and CDR3 of SEQ ID NO: 11; an isolated, purified or recombinant antibody or fragment thereof comprising a sequence of CDR1 of SEQ ID NO:1; CDR2 of SEQ ID NO:4; and CDR3 of SEQ ID NO: 12; an isolated, purified or recombinant antibody or fragment thereof comprising a sequence of CDR1 of SEQ ID NO:1; CDR2 of SEQ ID NO:4; and CDR3 of SEQ ID NO: 13; an isolated, purified or recombinant antibody or fragment thereof comprising a sequence of CDR1 of SEQ ID NO:1; CDR2 of SEQ ID NO:4; and CDR3 of SEQ ID NO: 14; an isolated, purified or recombinant antibody or fragment thereof comprising a sequence of CDR1 of SEQ ID NO:1; CDR2 of SEQ ID NO:4; and CDR3 of SEQ ID NO: 15; an isolated, purified or recombinant antibody or fragment thereof comprising a sequence of CDR1 of SEQ ID NO:1; CDR2 of SEQ ID NO:6; and CDR3 of SEQ ID NO: 10; an isolated, purified or recombinant antibody or fragment thereof comprising a sequence of CDR1 of SEQ ID NO:1; CDR2 of SEQ ID NO:6; and CDR3 of SEQ ID NO: 11; an isolated, purified or recombinant antibody or fragment thereof comprising a sequence of CDR1 of SEQ ID NO:1; CDR2 of SEQ ID NO:6; and CDR3 of SEQ ID NO: 12; an isolated, purified or recombinant antibody or fragment thereof comprising a sequence of CDR1 of SEQ ID NO:1; CDR2 of SEQ ID NO:6; and CDR3 of SEQ ID NO: 13; an isolated, purified or recombinant antibody or fragment thereof comprising a sequence of CDR1 of SEQ ID NO:1; CDR2 of SEQ ID NO:4; and CDR3 of SEQ ID NO: 16; an isolated, purified or recombinant antibody or fragment thereof comprising a sequence of CDR1 of SEQ ID NO:1; CDR2 of SEQ ID NO:4; and CDR3 of SEQ ID NO: 17; an isolated, purified or recombinant antibody or fragment thereof comprising a sequence of CDR1 of SEQ ID NO:1; CDR2 of SEQ ID NO:4; and CDR3 of SEQ ID NO: 18; an isolated, purified or recombinant antibody or fragment thereof comprising a sequence of CDR1 of SEQ ID NO:1; CDR2 of SEQ ID NO:6; and CDR3 of SEQ ID NO: 16; an isolated, purified or recombinant antibody or fragment thereof comprising a sequence of CDR1 of SEQ ID NO:1; CDR2 of SEQ ID NO:6; and CDR3 of SEQ ID NO: 17; an isolated, purified or recombinant antibody or fragment thereof comprising a sequence of CDR1 of SEQ ID NO:1; CDR2 of SEQ ID NO:6; and CDR3 of SEQ ID NO: 18.

The term “antibody”, also referred to in the art as “immunoglobulin” (Ig), used herein refers to a protein constructed from paired heavy and light polypeptide chains; various Ig isotypes exist, including IgA, IgD, IgE, IgG, and IgM. When an antibody is correctly folded, each chain folds into a number of distinct globular domains joined by more linear polypeptide sequences. For example, the immunoglobulin light chain folds into a variable (V_{L}) and a constant (C_{L}) domain, while the heavy chain folds into a variable (V_{H}) and three constant (C_{H}, C_{H2}, C_{H3}) domains. Interaction of the heavy and light chain variable domains (V_{H} and V_{L}) results in the formation of an antigen binding region (Fv). Each domain has a well-established structure familiar to those of skill in the art.

The light and heavy chain variable regions are responsible for binding the target antigen and can therefore show significant sequence diversity between antibodies. The constant regions show less sequence diversity, and are responsible for binding a number of natural proteins to elicit important biochemical events. The variable region of an antibody contains the antigen binding determinants of the molecule, and thus determines the specificity of an antibody for its target antigen. The majority of sequence variability occurs in six hypervariable regions, three each per variable heavy (V_{H}) and light (V_{L}) chain; the hypervariable regions combine to form the antigen-binding site, and contribute to binding and recognition of an antigenic determinant. The specificity and affinity of an antibody for its antigen is determined by the structure of the hypervariable regions, as well as their size, shape and chemistry of the surface they present to the antigen. Various schemes exist for identification of the regions of hypervariability, the two most common being those of Kabat and of Chothia and Lesk. Kabat et al (1991) define the “complementarity-determining regions” (CDR) based on sequence variability at the antigen-binding regions of the V_{H} and V_{L} domains. Chothia and Lesk (1987) define the “hypervariable loops” (H or L) based on the location of the structural loop regions in the V_{H} and V_{L} domains. As these individual schemes define CDR and hypervariable loop regions that are adjacent or overlapping, those of skill in the antibody art often utilize the terms “CDR” and “hypervariable loop” interchangeably, and they may be so used herein. For this reason, the regions forming the antigen-binding site are presently referred to herein as CDR L1, CDR L2, CDR L3, CDR H1, CDR H2, CDR H3 in the case of antibodies comprising a V_{H} and a V_{L} domain; or as CDR1, CDR2, CDR3 in the case of the antigen-binding regions of either a heavy chain or a light chain. The CDR/loops can also be referred to according to the IMGT numbering system (Lefranc et al, 2003), which was developed to facilitate comparison of variable domains. Additionally, a standardized delimitation of the framework regions and of the CDR is provided.

An “antibody fragment” as referred to herein may include any suitable antigen-binding antibody fragment known in the art. The antibody fragment may be a naturally-occurring antibody fragment, or may be obtained by manipulation of a naturally-occurring antibody or by using recombinant methods. For example, an antibody fragment may include, but is not limited to a Fv, single-chain Fv (scFv; a molecule consisting of V_{L} and V_{H} connected with a peptide linker), Fab, F(ab′)₂, single domain antibody (sdAb; a fragment composed of a single V_{L} or V_{H}), and multivalent presentations of any of these.

In a non-limiting example, the antibody fragment may be an sdAb derived from naturally-occurring sources. Heavy chain antibodies of camelid origin (Hamers-Casterman et al, 1993) lack light chains and thus their antigen binding sites consist of one domain, termed V_{H}H. sdAb have also been observed in shark and are termed V_{NAR} (Nuttall et al, 2003). Other sdAb may be engineered based on human Ig heavy and light chain sequences (Jespers et al, 2004; To et al, 2005). As used herein, the term “sdAb” includes those sdAb directly isolated from V_{H}, V_{H}H, V_{L}, or V_{NAR} reservoir of any origin through phage display or other technologies, sdAb derived from the aforementioned sdAb, recombinantly produced sdAb, as well as those sdAb generated through further modification of such sdAb by humanization, affinity maturation, stabilization, solubilization, e.g., camelization, or other methods of antibody engineering. Also encompassed by the present invention are homologues, derivatives, or fragments that retain the antigen-binding function and specificity of the sdAb.

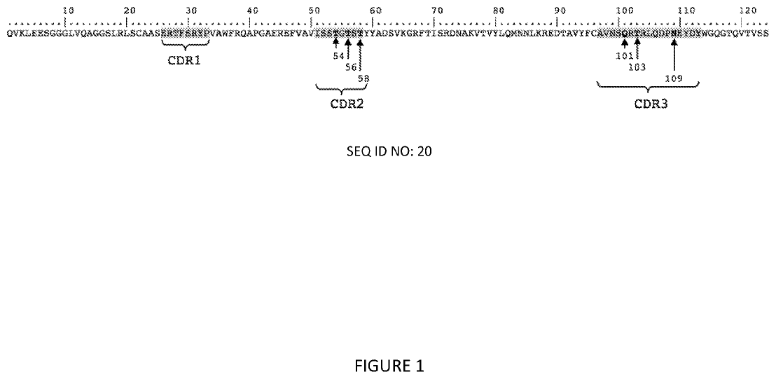

A person of skill in the art would be well-acquainted with the structure of a single-domain antibody (see, for example, 3DWT, 2P42 in Protein Data Bank). An sdAb comprises a single immunoglobulin domain that retains the immunoglobulin fold; most notably, only three CDR/hypervariable loops form the antigen-binding site. However, and as would be understood by those of skill in the art, not all CDR may be required for binding the antigen. For example, and without wishing to be limiting, one, two, or three of the CDR may contribute to binding and recognition of the antigen by the sdAb of the present invention. The CDR of the sdAb or variable domain are referred to herein as CDR1, CDR2, and CDR3, and delineated cording to the IMGT definition (Lefranc et al, 2003); however, to be consistent with the published structural data on the parental A26.8 V_{H}H (WO/2012/055030) (Murase et al, 2014) a sequential residue numbering system is used herein (FIG. 1).

As previously stated, the antibody or fragment thereof may be an sdAb. The sdAb may be of camelid origin or derived from a camelid V_{H}H, and thus may be based on camelid framework regions; alternatively, the CDR described above may be grafted onto V_{NAR}, V_{H}H, V_{H} or V_{L} framework regions. In yet another alternative, the hypervariable loops described above may be grafted onto the framework regions of other types of antibody fragments (Fv, scFv, Fab). The present embodiment further encompasses an antibody fragment that is “humanized” using any suitable method know in the art, for example, but not limited to CDR grafting and veneering. Humanization of an antibody or antibody fragment comprises replacing an amino acid in the sequence with its human counterpart, as found in the human consensus sequence, without loss of antigen-binding ability or specificity; this approach reduces immunogenicity of the antibody or fragment thereof when introduced into human subjects. In the process of CDR grafting, one or more than one of the heavy chain CDR defined herein may be fused or grafted to a human variable region (V_{H}, or V_{L}), or to other human antibody fragment framework regions (Fv, scFv, Fab). In such a case, the conformation of said one or more than one hypervariable loop is preserved, and the affinity and specificity of the sdAb for its target (i.e., toxins A and B) is also preserved. CDR grafting is known in the art and is described in at least the following: U.S. Pat. Nos. 6,180,370, 5,693,761, 6,054,297, 5,859,205, and European Patent No. 626390. Veneering, also referred to in the art as “variable region resurfacing”, involves humanizing solvent-exposed positions of the antibody or fragment; thus, buried non-humanized residues, which may be important for CDR conformation, are preserved while the potential for immunological reaction against solvent-exposed regions is minimized. Veneering is known in the art and is described in at least the following: U.S. Pat. Nos. 5,869,619, 5,766,886, 5,821,123, and European Patent No. 519596. Persons of skill in the art would also be amply familiar with methods of preparing such humanized antibody fragments and humanizing amino acid positions.

In a specific, non-limiting example, the antibody or fragment thereof that is specific for TcdA may comprise a sequence selected from the group consisting of:

QVKLEESGGGLVQAGGSLRLSCAASERTFSRYPVAWFRQAPGAEREFVAVISSX₁GX₂SX₃YYADSVKGRFTISRDNAKVTVYLQMNNLKREDTAVYFCAVNSX₄RX₅RLQDPX₆EYDYWGQGTQVTVSS (SEQ ID NO: 19); wherein: X₁ is T or R; X₂ is T, R or I; X₃ is T or K; X₄ is Q or K; X₅ is T, R, K, M or W; and X₆ is N or R; and combinations thereof; and excluding SEQ ID NO: 20.

Furthermore, the present invention provides an isolated, purified or recombinant antibody or fragment that may be selected from the group consisting of:

(SEQ ID NO: 21)
QVKLEESGGGLVQAGGSLRLSCAASERTFSRYPVAWFRQAPGAEREFVAV
ISSRGTSTYYADSVKGRFTISRDNAKVTVYLQMNNLKREDTAVYFCAVNS
QRTRLQDPNEYDYWGQGTQVTVSS;
(SEQ ID NO: 22)
QVKLEESGGGLVQAGGSLRLSCAASERTFSRYPVAWFRQAPGAEREFVAV
ISSTGRSTYYADSVKGRFTISRDNAKVTVYLQMNNLKREDTAVYFCAVNS
QRTRLQDPNEYDYWGQGTQVTVSS;
(SEQ ID NO: 23)
QVKLEESGGGLVQAGGSLRLSCAASERTFSRYPVAWFRQAPGAEREFVAV
ISSTGISTYYADSVKGRFTISRDNAKVTVYLQMNNLKREDTAVYFCAVNS
QRTRLQDPNEYDYWGQGTQVTVSS;
(SEQ ID NO: 24)
QVKLEESGGGLVQAGGSLRLSCAASERTFSRYPVAWFRQAPGAEREFVAV
ISSTGKSTYYADSVKGRFTISRDNAKVTVYLQMNNLKREDTAVYFCAVNS
QRTRLQDPNEYDYWGQGTQVTVSS;
(SEQ ID NO: 25)
QVKLEESGGGLVQAGGSLRLSCAASERTFSRYPVAWFRQAPGAEREFVAV
ISSTGTSTYYADSVKGRFTISRDNAKVTVYLQMNNLKREDTAVYFCAVNS
KRTRLQDPNEYDYWGQGTQVTVSS;
(SEQ ID NO: 26)
QVKLEESGGGLVQAGGSLRLSCAASERTFSRYPVAWFRQAPGAEREFVAV
ISSTGTSTYYADSVKGRFTISRDNAKVTVYLQMNNLKREDTAVYFCAVNS
QRRRLQDPNEYDYWGQGTQVTVSS;
(SEQ ID NO: 27)
QVKLEESGGGLVQAGGSLRLSCAASERTFSRYPVAWFRQAPGAEREFVAV
ISSTGTSTYYADSVKGRFTISRDNAKVTVYLQMNNLKREDTAVYFCAVNS
QRKRLQDPNEYDYWGQGTQVTVSS;
(SEQ ID NO: 28)
QVKLEESGGGLVQAGGSLRLSCAASERTFSRYPVAWFRQAPGAEREFVAV
ISSTGTSTYYADSVKGRFTISRDNAKVTVYLQMNNLKREDTAVYFCAVNS
QRMRLQDPNEYDYWGQGTQVTVSS;
(SEQ ID NO: 29)
QVKLEESGGGLVQAGGSLRLSCAASERTFSRYPVAWFRQAPGAEREFVAV
ISSTGTSTYYADSVKGRFTISRDNAKVTVYLQMNNLKREDTAVYFCAVNS
QRWRLQDPNEYDYWGQGTQVTVSS;
(SEQ ID NO: 30)
QVKLEESGGGLVQAGGSLRLSCAASERTFSRYPVAWFRQAPGAEREFVAV
ISSTGTSTYYADSVKGRFTISRDNAKVTVYLQMNNLKREDTAVYFCAVNS
QRTRLQDPREYDYWGQGTQVTVSS;
(SEQ ID NO: 31)
QVKLEESGGGLVQAGGSLRLSCAASERTFSRYPVAWFRQAPGAEREFVAV
ISSTGRSTYYADSVKGRFTISRDNAKVTVYLQMNNLKREDTAVYFCAVNS
KRTRLQDPNEYDYWGQGTQVTVSS;
(SEQ ID NO: 32)
QVKLEESGGGLVQAGGSLRLSCAASERTFSRYPVAWFRQAPGAEREFVAV
ISSTGRSTYYADSVKGRFTISRDNAKVTVYLQMNNLKREDTAVYFCAVNS
QRRRLQDPNEYDYWGQGTQVTVSS;
(SEQ ID NO: 33)
QVKLEESGGGLVQAGGSLRLSCAASERTFSRYPVAWFRQAPGAEREFVAV
ISSTGRSTYYADSVKGRFTISRDNAKVTVYLQMNNLKREDTAVYFCAVNS
QRKRLQDPNEYDYWGQGTQVTVSS;
(SEQ ID NO: 34)
QVKLEESGGGLVQAGGSLRLSCAASERTFSRYPVAWFRQAPGAEREFVAV
ISSTGRSTYYADSVKGRFTISRDNAKVTVYLQMNNLKREDTAVYFCAVNS
QRMRLQDPNEYDYWGQGTQVTVSS;
(SEQ ID NO: 35)
QVKLEESGGGLVQAGGSLRLSCAASERTFSRYPVAWFRQAPGAEREFVAV
ISSTGTSTYYADSVKGRFTISRDNAKVTVYLQMNNLKREDTAVYFCAVNS
KRRRLQDPNEYDYWGQGTQVTVSS;
(SEQ ID NO: 36)
QVKLEESGGGLVQAGGSLRLSCAASERTFSRYPVAWFRQAPGAEREFVAV
ISSTGTSTYYADSVKGRFTISRDNAKVTVYLQMNNLKREDTAVYFCAVNS
KRKRLQDPNEYDYWGQGTQVTVSS;
(SEQ ID NO: 37)
QVKLEESGGGLVQAGGSLRLSCAASERTFSRYPVAWFRQAPGAEREFVAV
ISSTGTSTYYADSVKGRFTISRDNAKVTVYLQMNNLKREDTAVYFCAVNS
KRMRLQDPNEYDYWGQGTQVTVSS;
(SEQ ID NO: 38)
QVKLEESGGGLVQAGGSLRLSCAASERTFSRYPVAWFRQAPGAEREFVAV
ISSTGRSTYYADSVKGRFTISRDNAKVTVYLQMNNLKREDTAVYFCAVNS
KRRRLQDPNEYDYWGQGTQVTVSS;
(SEQ ID NO: 39)
QVKLEESGGGLVQAGGSLRLSCAASERTFSRYPVAWFRQAPGAEREFVAV
ISSTGRSTYYADSVKGRFTISRDNAKVTVYLQMNNLKREDTAVYFCAVNS
KRKRLQDPNEYDYWGQGTQVTVSS;
(SEQ ID NO: 40)
QVKLEESGGGLVQAGGSLRLSCAASERTFSRYPVAWFRQAPGAEREFVAV
ISSTGRSTYYADSVKGRFTISRDNAKVTVYLQMNNLKREDTAVYFCAVNS
KRMRLQDPNEYDYWGQGTQVTVSS; or a sequence
substantially identical thereto.

A substantially identical sequence may comprise one or more conservative amino acid mutations. It is known in the art that one or more conservative amino acid mutations to a reference sequence may yield a mutant peptide with no substantial change in physiological, chemical, physico-chemical or functional properties compared to the reference sequence; in such a case, the reference and mutant sequences would be considered “substantially identical” polypeptides. Conservative amino acid mutation may include addition, deletion, or substitution of an amino acid; a conservative amino acid substitution is defined herein as the substitution of an amino acid residue for another amino acid residue with similar chemical properties (e.g. size, charge, or polarity).

In a non-limiting example, a conservative mutation may be an amino acid substitution. Such a conservative amino acid substitution may substitute a basic, neutral, hydrophobic, or acidic amino acid for another of the same group. By the term “basic amino acid” it is meant hydrophilic amino acids having a side chain pK value of greater than 7, which are typically positively charged at physiological pH. Basic amino acids include histidine (His or H), arginine (Arg or R), and lysine (Lys or K). By the term “neutral amino acid” (also “polar amino acid”), it is meant hydrophilic amino acids having a side chain that is uncharged at physiological pH, but which has at least one bond in which the pair of electrons shared in common by two atoms is held more closely by one of the atoms. Polar amino acids include serine (Ser or S), threonine (Thr or T), cysteine (Cys or C), tyrosine (Tyr or Y), asparagine (Asn or N), and glutamine (Gln or Q). The term “hydrophobic amino acid” (also “non-polar amino acid”) is meant to include amino acids exhibiting a hydrophobicity of greater than zero according to the normalized consensus hydrophobicity scale of Eisenberg (1984). Hydrophobic amino acids include proline (Pro or P), isoleucine (Ile or I), phenylalanine (Phe or F), valine (Val or V), leucine (Leu or L), tryptophan (Trp or W), methionine (Met or M), alanine (Ala or A), and glycine (Gly or G). “Acidic amino acid” refers to hydrophilic amino acids having a side chain pK value of less than 7, which are typically negatively charged at physiological pH. Acidic amino acids include glutamate (Glu or E), and aspartate (Asp or D).

Sequence identity is used to evaluate the similarity of two sequences; it is determined by calculating the percent of residues that are the same when the two sequences are aligned for maximum correspondence between residue positions. Any known method may be used to calculate sequence identity; for example, computer software is available to calculate sequence identity. Without wishing to be limiting, sequence identity can be calculated by software such as NCBI BLAST2 service maintained by the Swiss Institute of Bioinformatics (and as found at http://ca.expasy.org/tools/blast/), BLAST-P, Blast-N, or FASTA-N, or any other appropriate software that is known in the art.

The substantially identical sequences of the present invention may be at least 65% identical; in another example, the substantially identical sequences may be at least 65, 70, 85, 90, 95, 96, 97, 98, 99, or 100% identical, or any percentage therebetween, at the amino acid level to sequences described herein. Importantly, the substantially identical sequences retain the activity and specificity of the reference sequence. In a non-limiting embodiment, the difference in sequence identity may be due to conservative amino acid mutation(s). By way of example only, and without wishing to be limiting in any manner, the V_{H}Hs of the present invention have between about 66% and 82% sequence identity (see Tables 5 and 6). In another non-limiting example, the present invention may be directed to an antibody or fragment thereof comprising a sequence at least 98% identical to that of the V_{H}Hs described herein.

The isolated or purified antibody or fragment thereof of the present invention may bind to a conformational or linear epitope. A conformational epitope is formed by amino acid residues that are discontinuous in sequence, but proximal in the three-dimensional structure of the antigen. In contrast, a linear epitope (also referred to in the art as a “sequential epitope”) is recognized by its linear amino acid sequence, or primary structure. The conformational and linear epitopes of the antibodies or fragments thereof of the present invention recognize conformational and linear epitopes located in the region of TcdA responsible for cell-receptor binding.

The antibody or fragment thereof of the present invention may also comprise additional sequences to aid in expression, detection or purification of a recombinant antibody or fragment thereof. Any such sequences or tags known to those of skill in the art may be used. For example, and without wishing to be limiting, the antibody or fragment thereof may comprise a targeting or signal sequence (for example, but not limited to ompA), a detection/purification tag (for example, but not limited to c-Myc or a His₅ or His₆), or a combination thereof. In another example, the additional sequence may be a biotin recognition site such as that described by Cronan et al in WO 95/04069 or Voges et al in WO/2004/076670. As is also known to those of skill in the art, linker sequences may be used in conjunction with the additional sequences or tags, or may serve as a detection/purification tag.

The antibody or fragment thereof of the present invention may also be in a multivalent display format, also referred to herein as multivalent presentation. Multimerization may be achieved by any suitable method of know in the art. For example, and without wishing to be limiting in any manner, multimerization may be achieved using self-assembly molecules as described in Zhang et al (2004a; 2004b) and WO2003/046560. The described method produces pentabodies by expressing a fusion protein comprising the antibody or fragment thereof of the present invention and the pentamerization domain of the B-subunit of an AB₅ toxin family (Merritt & Hol, 1995); the pentamerization domain assembles into a pentamer, through which a multivalent display of the antibody or fragment thereof is formed. Each subunit of the pentamer may be the same or different, and may have the same or different specificity. Additionally, the pentamerization domain may be linked to the antibody or antibody fragment using a linker; such a linker should be of sufficient length and appropriate composition to provide flexible attachment of the two molecules, but should not hamper the antigen-binding properties of the antibody.

Other forms of multivalent display are also encompassed by the present invention. For example, and without wishing to be limiting, the antibody or fragment thereof may be presented as a dimer, a trimer, or any other suitable oligomer. This may be achieved by methods known in the art, for example direct linking connection (Nielson et al, 2000), c-jun/Fos interaction (de Kruif & Logtenberg, 1996), “Knob into holes” interaction (Ridgway et al, 1996). Another method known in the art for multimerization is to dimerize the antibody or fragment thereof using an Fc domain, e.g., human Fc domains. The Fc domains may be selected from various classes including, but not limited to, IgG, IgM, or various subclasses including, but not limited to IgG1, IgG2, etc. In this approach, the Fc gene in inserted into a vector along with the sdAb gene to generate a sdAb-Fc fusion protein (Bell et al, 2010; Iqbal et al, 2010); the fusion protein is recombinantly expressed then purified. For example, and without wishing to be limiting in any manner, multivalent display formats may encompass chimeric formats of anti-TcdA V_{H}Hs linked to an Fc domain, or bi- or tri-specific antibody fusions with two or three anti-TcdA V_{H}Hs recognizing unique epitopes. Enhanced toxin neutralizing efficacy may also be obtained using various techniques, including PEGylation, fusion to serum albumin, or fusion to serum albumin-specific antibody fragments; these approaches increase their blood circulation half lives, size and avidity.

The present invention also encompasses nucleic acid sequences encoding the molecules as described herein. The nucleic acid sequence may be codon-optimized for expression in various micro-organisms. The present invention also encompasses vectors comprising the nucleic acids as just described. Furthermore, the invention encompasses cells comprising the nucleic acid and/or vector as described.

The present invention further encompasses the isolated or purified antibody or fragments thereof immobilized onto a surface using various methodologies; for example, and without wishing to be limiting, the antibody or fragment may be linked or coupled to the surface via His-tag coupling, biotin binding, covalent binding, adsorption, and the like. Immobilization of the antibody or fragment thereof of the present invention may be useful in various applications for capturing, purifying or isolating proteins. The solid surface may be any suitable surface, for example, but not limited to the well surface of a microtiter plate, channels of surface plasmon resonance (SPR) sensorchips, membranes, beads (such as magnetic-based or sepharose-based beads or other chromatography resin), glass, plastic, stainless steel, a film, or any other useful surface such as nanoparticles, nanowires and cantilever surfaces.

Thus, the present invention also provides a method of capturing Clostridium difficile toxins, comprising contacting a sample (such as, but not limited to C. difficile culture supernatant, human/animal intestinal/colonic fluid, or any other suitable sample) with one or more than one isolated or purified antibody or fragment thereof of the present invention. The isolated or purified antibody or fragments thereof may be immobilized onto a surface. The toxin(s) then bind to the isolated or purified antibody or fragment thereof and are thus captured. The toxins may then optionally be identified by mass spectrometric methods and/or released or eluted from their interaction with the antibody or fragment thereof; methods for releasing or eluting bound molecules are well-known to those of skill in the art (for example but not limited to heat elution steps), as are spectrometric methods capable of detecting and identifying the toxin. The isolated or purified antibody or fragment thereof of the present invention provide particularly robust affinity purification reagents due to their resistance to acidic and heat elution steps.

The invention also encompasses the antibody or fragment thereof as described above linked to a cargo molecule. The cargo molecule may be any suitable molecule. For example, and without wishing to be limiting in any manner, the cargo molecule may be a detectable agent, a therapeutic agent, a drug, a peptide, an enzyme, a protease, a carbohydrate moiety, a cytotoxic agent, one or more liposomes loaded with any of the previously recited types of cargo molecules, or one or more nanoparticle, nanowire, nanotube, or quantum dots. For example, and without wishing to be limiting in any manner, the cargo molecule may be a protease that may digest the C. difficile toxin; in a further non-limiting example, the protease may be linked to a V_{H}H such as a mutant V_{H}H that is protease resistant. In yet another non-limiting example, the cargo molecule may be a cytotoxic agent that may be antibacterial or toxic towards host cells “infected” with C. difficile toxins. In a further non-limiting example, the cargo molecule is a liposome, which makes the construct well-suited as a delivery agent for mucosal vaccines. The cargo molecule may be linked to the antibody or fragment thereof by any suitable method known in the art. For example, and without wishing to be limiting, the cargo molecule may be linked to the peptide by a covalent bond or ionic interaction. The linkage may be achieved through a chemical cross-linking reaction, or through fusion using recombinant DNA methodology combined with any peptide expression system, such as bacteria, yeast or mammalian cell-based systems. Methods for linking an antibody or fragment thereof to a therapeutic agent or detectable agent would be well-known to a person of skill in the art.

The present invention also encompasses an antibody or fragment thereof linked to a detectable agent. For example, the TcdA-specific antibody or fragment thereof may be linked to a radioisotope, a paramagnetic label, a fluorophore, an affinity label (for example biotin, avidin, etc), fused to a detectable protein-based molecule, nucleotide, quantum dot, nanoparticle, nanowire, or nanotube or any other suitable agent that may be detected by imaging methods. In a specific, non-limiting example, the antibody or fragment thereof may be linked to a fluorescent agent such as FITC or may genetically be fused to the Enhanced Green Fluorescent Protein (EGFP). The antibody or fragment thereof may be linked to the detectable agent using any method known in the art (recombinant technology, chemical conjugation, etc.).

Thus, the present invention further provides a method of detecting Clostridium difficile toxins, comprising contacting a sample (such as, but not limited to C. difficile culture supernatant, human/animal intestinal/colonic fluid, or any other suitable sample) with one or more than one isolated or purified antibody or fragment thereof of the present invention. The isolated or purified antibody or fragments thereof may be linked to a detectable agent. The toxin(s) can then be detected using detection and/or imaging technologies known in the art, such as, but not limited to mass spectrometric or immunoassay methods.

For example, and without wishing to be limiting in any manner, the isolated or purified antibody or fragment thereof linked to a detectable agent may be used in immunoassays (IA) including, but not limited to enzyme IA (EIA), ELISA, “rapid antigen capture”, “rapid chromatographic IA”, and “rapid EIA”. For examples, see Planche et al, 2008; Sloan et al, 2008; Rüssmann et al, 2007; Musher et al, 2007; Turgeon et al, 2003; Fenner et al, 2008.

The present invention also encompasses the isolated or purified antibody or fragment thereof for detection of toxins in neutralized cell toxicity assays; methods for cell toxicity assays, also referred to herein as cytotoxicity assays, are known in the art and include, but are not limited to those described by Planche et al (2008); Musher et al (2007); Turgeon et al (2003); and Fenner et al (2008). Cell cytotoxicity assays involve incubating samples (for example, but not limited to patient stool samples) with cultured cells (for example, but not limited to fibroblasts) alone, or with the addition of a neutralizing agent, in this case, the isolated or purified antibody or fragment thereof as described herein. If the presence of the neutralizing agent reduces or eliminates cell toxicity observed with the cultured cells alone, presence of the toxins in the sample is confirmed. This type of assay is the practical gold standard for CDAD detection in hospital diagnostic laboratories.

The present invention also encompasses a composition comprising one or more than one isolated or purified antibody or fragment thereof as described herein. The composition may comprise a single antibody or fragment as described above, or may be a mixture of antibodies or fragments.

The composition may also comprise a pharmaceutically acceptable diluent, excipient, or carrier. The diluent, excipient, or carrier may be any suitable diluent, excipient, or carrier known in the art, and must be compatible with other ingredients in the composition, with the method of delivery of the composition, and is not deleterious to the recipient of the composition. The composition may be in any suitable form; for example, the composition may be provided in suspension form, powder form (for example, but limited to lyophilised or encapsulated), capsule or tablet form. For example, and without wishing to be limiting, when the composition is provided in suspension form, the carrier may comprise water, saline, a suitable buffer, or additives to improve solubility and/or stability; reconstitution to produce the suspension is effected in a buffer at a suitable pH to ensure the viability of the antibody or fragment thereof. Dry powders may also include additives to improve stability and/or carriers to increase bulk/volume; for example, and without wishing to be limiting, the dry powder composition may comprise sucrose or trehalose. In a specific, non-limiting example, the composition may be so formulated as to deliver the antibody or fragment thereof to the gastrointestinal tract of the subject. Thus, the composition may comprise encapsulation, time-release, or other suitable technologies for delivery of the antibody or fragment thereof. It would be within the competency of a person of skill in the art to prepare suitable compositions comprising the present compounds.

The present invention also comprises a method of treating a Clostridium difficile infection, comprising administering the isolated or purified antibody or fragment thereof of the present invention, or a composition comprising the antibody or fragment thereof, to a subject in need thereof. Any suitable method of delivery may be used. For example, and without wishing to be limiting in any manner, the antibody or fragment thereof, or the composition, may be delivered systemically (orally, nasally, intravenously, etc.) or may be delivered to the gastrointestinal tract. Those of skill in the art would be familiar with such methods of delivery.

The present invention will be further illustrated in the following examples. However, it is to be understood that these examples are for illustrative purposes only and should not be used to limit the scope of the present invention in any manner.

### EXAMPLE 1

### In Silico Affinity Maturation

The atomic coordinates of the A26.8 V_{H}H (WO/2012/055030) bound to the C-terminal portion of TcdA were taken from the structure of the A26.8H6-TcdA-A2 complex crystallized at pH 6.5 (PDB ID: 4NC0), (Murase et al, 2014) and was used as starting point for virtual affinity maturation. Two versions of the complex were prepared, differing by exclusion (preparation 1) or inclusion (preparation 2) of the C-terminal G262 of TcdA and N-terminal Q1 of the V_{H}H, which are not visible in the crystal structure but may affect the calculated interactions in the complex. All TcdA amino-acid residues preceding T123, which are distant form the V_{H}H, and the His-tag residue H125 at the C-terminus of the V_{H}H, were deleted from the crystal structure. To be consistent with the published structural data (Murase et al, 2014) a sequential residue numbering system is used in this paper. Hydrogen atoms were added to the resulting complex and adjusted for maximizing H-bonding interactions. Structural refinement of the complex was then carried out by energy-minimization using the AMBER force-field (Hornak et al, 2006; Cornell et al, 1995) with a distance-dependent dielectric and infinite cutoff for non-bonded interactions. Non-hydrogen atoms were restrained at their crystallographic positions with harmonic force constants of 40 and 10 kcal/(mol·A²) for the backbone and side-chain atoms, respectively.

The ADAPT platform was then used for affinity maturation (Vivcharuk et al, 2017). In the first round of affinity optimization, single-point scanning mutagenesis simulations were carried out at several positions within the CDRs of A26.8 V_{H}H. We used three protocols: SIE-SCWRL

(Krivov et al, 2009; Naim et al, 2007; Sulea and Purisima, 2012), FoldX (Guerois et al, 2002; Schymkowitz et al, 2005), and Rosetta (O'Conchuir et al, 2015; Rohl et al, 2004), for building the structures and evaluating the energies of single-point mutations to 17 other possible natural amino acids (Cys and Pro were excluded) at these positions of the parental sequence. A consensus approach over specific versions of these three protocols was applied for building and scoring the V_{H}H mutants. Scoring was mainly based on the consensus Z-score and also on the average rank score over the scores calculated with component methods. Further technical and implementation details of this approach and its component methods can be found in (Sulea et al, 2016) Substitutions predicted to introduce predicted folding free energy changes larger than 2.71 kcal/mol (i.e., 100-fold increase of unfolding equilibrium constant) relative to the parental molecule were discarded from further evaluation. In the second round of optimization, double- and triple-point V_{H}H mutants were generated from combinations of the lead single-point mutations selected after experimental validation, and scored using the same computational protocol as for the single-point mutants.

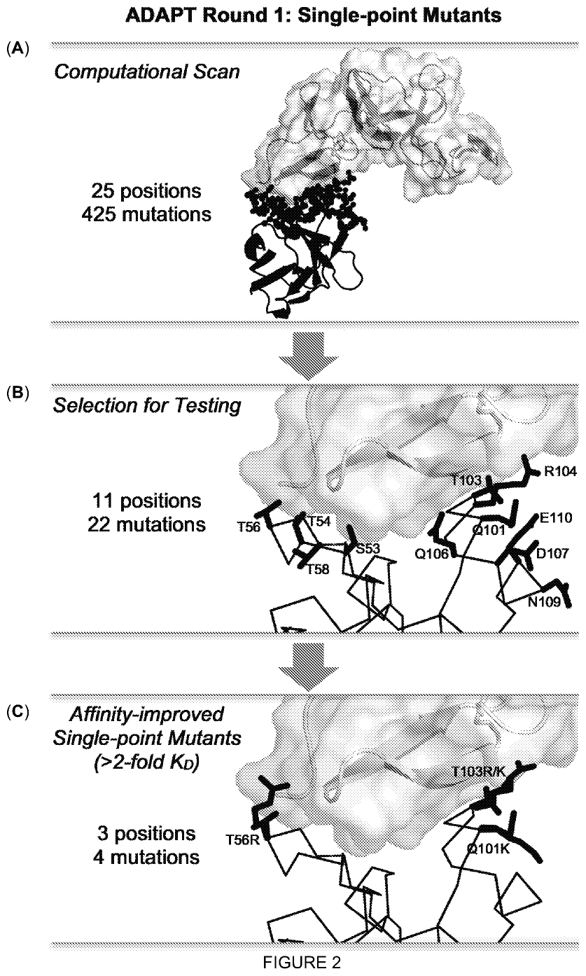

In the first round of ADAPT, 425 single-point mutations to all natural amino-acids except Cys and Pro were computationally evaluated in the CDRs of A26.8 V_{H}H. The scanned region covered 25 positions (S30-P33 from CDR1, V50-Y59 from CDR2, and S100-E110 from CDR3) that when substituted have the potential to alter the antigen-binding affinity. The proximity of the targeted residues to the TcdA fragment can be seen in FIG. 2A.

The selection of the most likely single-point mutants with improved antigen-binding affinities was primarily guided by the top 50 consensus Z-scores based on predictions from the three computational methods within ADAPT (TABLE 1). Additional information was gleaned from the top 50 single-point mutations sorted by the average rank of the three component methods of ADAPT. Although these largely overlap with the mutations ranked by the consensus Z-score, there are several differences with the notable addition of 2 new positions (T58 and N109).

TABLE 1
Top 50 consensus Z-scores for single mutants.

| | | | | | | | | | | | | | | | | | | |
|---|---|---|---|---|---|---|---|---|---|---|---|---|---|---|---|---|---|---|
| Residue | R | K | Q | N | S | T | H | W | Y | F | M | L | I | V | A | G | E | D |

| | | | | | | | | | | | | | | | | | |
|---|---|---|---|---|---|---|---|---|---|---|---|---|---|---|---|---|---|
| R104 | | | −1.0 | −1.3 | | | | −5.2 | −3.3 | −3.4 | | −0.9 | | | | | |
| Q106 | | | | | | | | −1.9 | | −1.2 | | −1.4 | −0.9 | | | | −0.9 |
| T56 | −1.7 | | | | | | | | | | | | −1.2 | | | | |
| T103 | −1.5 | −1.5 | −1.0 | | | | | −1.6 | | | | | | | | | |
| T54 | −1.6 | | | | | | | −1.4 | −1.1 | −1.1 | | | | | | | |
| D107 | −1.5 | −1.1 | −1.3 | −1.1 | −1.1 | −1.1 | −1.2 | −1.4 | −1.5 | −1.5 | −1.2 | −1.2 | −1.2 | −1.2 | −1.1 | −1.1 | −1.3 |
| Y59 | | | | | | | | | | | | −1.5 | | | | | |
| P108 | −1.0 | −1.1 | | | | | | −1.0 | −1.0 | −1.0 | −1.0 | | | | −0.9 | | |
| S57 | | | | −1.0 | | | | | | | | | | | | | |
| E110 | | | −1.0 | | | | | | | | | | | | | | |
| Q101 | | | | | −0.9 | | | | | | | | | | | | |
| S53 | −0.9 | | | | | | | | | | | | | | | | |

Structure preparation 2 was used, which includes modeled N-terminal residue Q1 of V_{H}H and C-terminal residue G262 of TcdA. Single-point mutations experimentally tested are highlighted in bold. Parent antibody Z-score = −0.33.

Visual examination of the molecular interactions predicted with the three sampling protocols of ADAPT (SCWRL, Rosetta, FoldX), in terms of antibody-antigen interaction complementarity and overall structural geometry, narrowed down this selection to a set of 22 single-point mutations at 11 positions for further evaluation. These point mutations can be classified in two groups according to their location relative to the antigen and the interaction type (TABLE 2). The “Contact” set includes 15 substitutions at 6 positions contributing to the main contact surface interacting with the antigen, whereas the 7 mutations forming the “Peripheral” set substitute 5 positions located more at the periphery of the antibody-antigen interface (FIG. 2B). The mutation T103M was not listed in the top-50 mutations by either the consensus Z-score or average rank, but was selected based on a favorable FoldX binding affinity score and the prediction of good structural complementarity to the antigen surface.

TABLE 2
Summary of the ADAPT-based selection for single-point mutants
of A26.8 V_{H}H with predicted increased affinity for toxin A. ^{a}

| Mutation | | |
|---|---|---|
| group | CDR2 mutations | CDR3 mutations |
| Contact | S53R | T103R, T103K, T103W, T103M ^{c} |
| | T54R, T54W, T54Y | R104W |
| | T56R, T56I | Q106W, Q106F, Q106Y, Q106L |
| Peripheral | T58K ^{b} | Q101K ^{b} |
| | | D107R, D107Y, D107N |
| | | N109R ^{b} |
| | | E110Q |

^{a} From top-50 consensus Z-scores and visual inspection unless otherwise specified.
^{b} From top-50 average rank scores and visual inspection.
^{c} Based on the FoldX binding score and visual inspection.

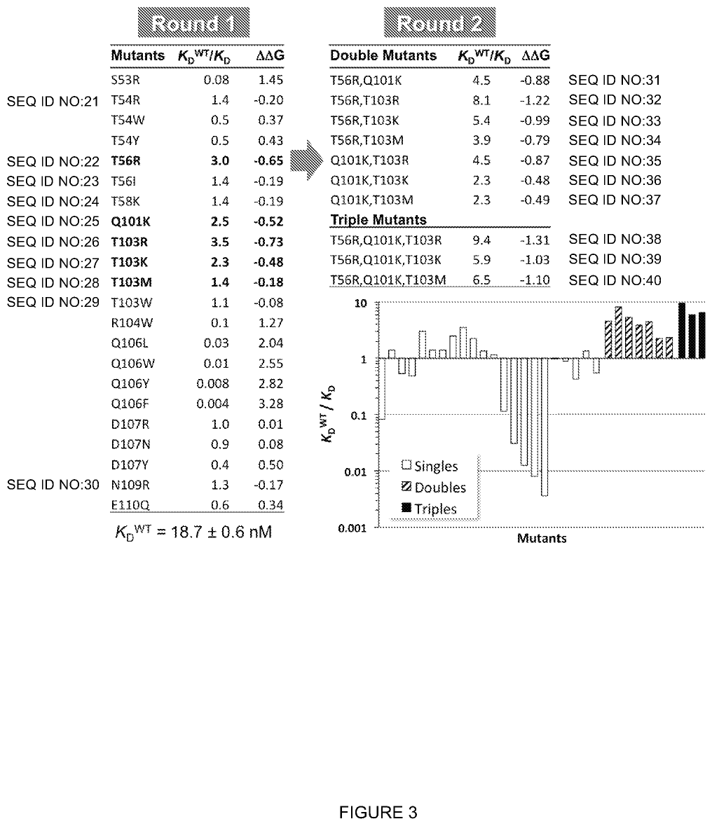

The four lead single-point mutations from round 1, possessing greater than 2-fold improvements in K_{D}s as determined experimentally (see Example 3 and FIG. 3), were carried forward to round 2 of affinity maturation. These include T56R in CDR2, and Q101K, T103R and T103K in CDR3. Combination of these mutations can lead to a total of 5 double mutants and 2 triple mutants. Because of the proximity of positions 101 and 103 that are both substituted with positively-charged amino acids, we also included in this round the charge-neutral mutation T103M which led to a slight affinity improvement in the first round, hence adding 2 double and 1 triple mutants to the second set. These 10 mutants considered in the second round were first verified computationally within ADAPT, mainly for potential changes in folding stability as a result of adjacent mutations. All mutants were predicted to have either improved or similar stabilities relative to the parental V_{H}H. Relative changes in binding affinity were also calculated with the ADAPT protocols, which generally predicted additive improvements relative to the component single mutations. Exceptions included the non-additivity predicted for the double- and triple-mutants that had simultaneous introduction of two basic residues in CDR3 (Q101K and T103R/K); nevertheless these mutations were predicted to improve affinity relative to the parent molecule. In these cases, introduction of Met at position 101 was predicted to restore some additivity and hence supported its consideration for the second round of optimization.

### EXAMPLE 2

### Protein Expression and Purification

The DNA sequences of A26.8 V_{H}H mutants were synthesized commercially by Thermo-Fisher/GENEART (Regensburg, Germany), subcloned into the pSJF2H expression vector (Arbabi-Ghahroudi et al, 2009), with C-terminal Myc/His6 tags, and were subsequently expressed in E. coli. Briefly, 50 ng of plasmid DNA was added to 5 μL of Mix and Go TG1 E. coli competent cells (Zymo Research, Irvine, Calif.) according to the manufacturer's instructions before plating onto 2×YT/ampicillin agar plates and incubation overnight at 32° C. For expression, 5 mL 2×YT cultures containing 100 μg/mL ampicillin, 1% (w/v) glucose were inoculated with single plasmid-bearing E. coli colonies and grown overnight at 37° C. with shaking at 220 rpm. The next day, 1 mL of overnight culture was inoculated into 250 mL 2×YT/ampicillin, 0.1% (w/v) glucose, in 500 mL baffled Ultra Yield flasks (Thomson Instruments Inc., Oceanside, Calif.) with air top seals and grown until an OD₆₀₀˜0.5-0.8. Cultures were then induced with 200 mM IPTG and grown overnight at 37° C. with shaking at 220 rpm. Periplasmic-targeted V_{H}Hs were extracted by osmotic shock and supernatants filtered through 0.22 μM filters (Millipore, Etobicoke, ON, Canada). V_{H}Hs were purified by immobilized metal-ion affinity chromatography (IMAC) using Ni Sepharose™ excel affinity resin (GE Healthcare, Mississauga, ON, Canada) in binding buffer containing PBS pH 7.4, 400 mM NaCl, and eluted in buffer containing PBS pH 7.4, 400 mM NaCl, 250 mM imidazole.

### EXAMPLE 3

### Binding Affinity Measurements

Before surface plasmon resonance (SPR) binding experiments, IMAC-purified V_{H}Hs were subjected to de-salting and further purification by size-exclusion chromatography (SEC). Approximately 500 μg of each V_{H}H was injected over a Superdex 75 Increase 10/300 GL SEC column (GE Healthcare) in Biacore running buffer HBS-EP (10 mM HEPES pH 7.4, 150 mM NaCl, 3 mM EDTA, 0.005% (v/v) P20; GE Healthcare) under the control of an ÄKTA FPLC at 0.8 mL/min. Monomeric V_{H}H fractions were collected and analyzed for binding to TcdA using a Biacore 3000 SPR instrument (GE Healthcare). Approximately 4,500 resonance units (RUs) of TcdA (List Biological Laboratories, Campbell, Calif.) were immobilized on a CM5 sensor chip (GE Healthcare) using the conditions previously described (Hussack et al, 2011). HBS-EP running buffer was used for all binding studies and regeneration of TcdA surfaces. Next, various dilution ranges of V_{H}Hs (as low as 10 nM-0.5 nM to as high as 5 μM-50 nM) were injected over immobilized TcdA at a flow rate of 40 μL/min with 120 s contact time and 600 s dissociation. Reference-subtracted sensorgrams were analyzed with BIAevaluation software (GE Healthcare) and fit to a 1:1 binding model. In cases where rapid kₒₙ and k_{off} rate constants were observed, equilibrium dissociation constants (K_{D}s) were determined by steady-state analysis. All mutant A26.8 V_{H}Hs were run in duplicates and the parent A26.8 V_{H}H in quintuplicate.

The 22 single-point mutants selected by ADAPT were produced and tested comparatively with the parental 26.8 V_{H}H. The K_{D}s and kinetic constants (kₒₙ and k_{off}) measured by SPR are listed in TABLE 3. Improvements in binding affinities relative to the parent A26.8 V_{H}H are presented in FIG. 3. Almost half of the single-point mutants demonstrate improved binding affinities, with two mutants (T56R and T103R) showing about a 3-fold improvement, and another two (Q101K and T103K) over 2-fold. The location of these mutations relative to the antigen epitope is illustrated in FIG. 2C. Substitution of T103 with the hydrophobic residues Met and Trp led to smaller improvements than with Arg and Lys. Similarly, a smaller improvement was obtained by substituting T56 with Ile rather than Arg. Mutations T58K and N109R, considered based on their average rank scores, only marginally improved antigen binding. The same marginal improvement was also observed for the T54R mutation; however, T54 substitution with aromatic amino acids decreased affinity. However, a number of mutations were predicted to improve antigen-binding affinity by in silico calculations, but instead had the opposite effect of weakening binding affinity. Such false-positive hits included mutations at positions D107 and E110 (up to 2.5-fold decrease in affinity), at S53 and R104 (about 10-fold decrease in affinity) and at Q106 (30-300-fold decrease in affinity). These results demonstrate that although the ADAPT computational protocol provided a reasonable focusing of the mutation space towards some of the actual affinity-maturation hot spots, the protocol was unable to distinguish these hot spots from other false-positive mutations. Hence, it was unobvious which Round-1 mutations will in fact lead to binding affinity and activity improvements without experimental testing the entire larger pool of single mutants suggested by ADAPT as potential hits.

TABLE 3
Binding and stability analyses of A26.8 V_{H}H mutants.

| | kₒₙ (10⁶) | k_{off} (10⁻²) | | |
|---|---|---|---|---|
| V_{H}H | (M⁻¹s⁻¹) ^{a} | (s⁻¹) ^{a} | K_{D} (nM) ^{a} | Tₘ (° C.) ^{b} |
| Parent A26.8 | 1.81 (0.32) | 3.39 (0.57) | 18.7 (0.6) | 78.2 (0.1) |
| Round 1 mutants | | | | |
| T103R | 2.70 (0.97) | 1.40 (0.29) | 5.32 (0.82) | 79.1 (0.1) |
| T56R | 4.75 (2.84) | 2.69 (0.96) | 6.16 (1.68) | 77.2 (0.1) |
| Q101K | 2.70 (0.91) | 1.98 (0.18) | 7.63 (1.89) | 81.4 (0.1) |
| T103K | 2.47 (1.05) | 1.99 (0.57) | 8.29 (1.18) | 78.5 (0.1) |
| T54R | 3.55 (1.77) | 4.40 (1.07) | 13.2 (3.6) | 75.5 (0.0) |
| T58K | 2.47 (0.65) | 3.27 (0.33) | 13.5 (2.3) | 76.9 (0.2) |
| T56I | 1.94 (0.47) | 2.62 (0.45) | 13.6 (1.0) | 76.5 (0.0) |
| T103M | 1.77 (0.52) | 2.37 (0.45) | 13.7 (1.5) | 77.4 (0.1) |
| N109R | 1.95 (0.23) | 2.68 (0.01) | 13.9 (1.6) | 77.6 (0.1) |
| T103W | 2.39 (1.04) | 3.61 (0.39) | 16.3 (5.5) | 77.5 (0.0) |
| D107R | 1.39 (0.27) | 2.59 (0.06) | 19.0 (3.3) | 70.8 (0.5) |
| D107N | 1.92 (0.47) | 4.02 (0.62) | 21.3 (2.1) | 74.9 (0.1) |
| E110Q | 2.08 (0.38) | 6.99 (1.64) | 33.6 (1.8) | 78.1 (0.1) |
| T54W | 1.61 (0.17) | 5.61 (0.01) | 35.0 (3.7) | 74.0 (0.6) |
| T54Y | 1.81 (0.37) | 6.94 (0.21) | 38.9 (6.8) | 76.0 (0.1) |
| D107Y | 1.62 (0.26) | 7.11 (0.93) | 44.0 (1.3) | 73.4 (0.1) |
| R104W | 0.33 (0.03) | 5.48 (0.77) | 165 (9) | 77.3 (0.0) |
| S53R ^{c} | | | 224 (170) | 74.0 (0.0) |
| Q106L ^{c} | | | 612 (53) | 80.0 (0.0) |
| Q106W ^{c} | | | 1475 (177) | 79.7 (0.1) |
| Q106Y ^{c} | | | 2315 (473) | 79.0 (0.0) |
| Q106F ^{c} | | | 5130 (1174) | 81.0 (0.0) |
| Round 2 mutants | | | | |
| T56R, Q101K, T103R | 12.3 (4.56) | 2.41 (0.81) | 1.98 (0.08) | 81.6 (0.1) |
| T56R, T103R | 8.33 (0.87) | 1.93 (0.17) | 2.32 (0.04) | 78.5 (0.1) |
| T56R, Q101K, T103M | 10.4 (1.95) | 2.96 (0.22) | 2.87 (0.33) | 80.9 (0.1) |
| T56R, Q101K, T103K | 14.7 (3.61) | 4.67 (1.06) | 3.19 (0.06) | 81.3 (0.1) |
| T56R, T103K | 8.61 (3.25) | 2.91 (0.85) | 3.44 (0.30) | 78.5 (0.1) |
| T56R, Q101K | 12.4 (3.18) | 4.97 (0.28) | 4.13 (0.84) | 81.4 (0.1) |
| Q101K, T103R | 5.45 (0.65) | 2.26 (0.24) | 4.20 (0.95) | 81.9 (0.1) |
| T56R, T103M | 6.42 (1.09) | 3.04 (0.33) | 4.84 (1.34) | 77.0 (0.0) |
| Q101K, T103M | 3.31 (0.08) | 2.65 (0.13) | 8.02 (0.57) | 81.3 (0.0) |
| Q101K, T103K | 4.96 (1.33) | 3.96 (0.26) | 8.21 (1.68) | 81.5 (0.0) |

^{a} Mean (±standard deviation) from n = 2 experiments, except for the parent V_{H}H (n = 5).
^{b} Mean (±standard deviation) from n = 4 samples.
^{c} K_{D}s determined by steady-state affinity analysis.

In this first ADAPT round consisting of single-point mutants, there was no clear trend as to whether k_{off} or kₒₙ was more affected by the mutations. For example, in the case of the best two single-point mutants, T103R seems to benefit from an improvement in k_{off} whereas T56R from an improvement in kₒₙ (about 2.5-fold relative to the parent in each case).

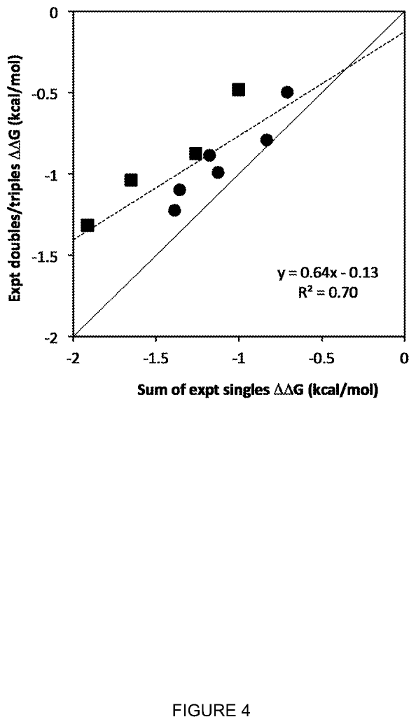

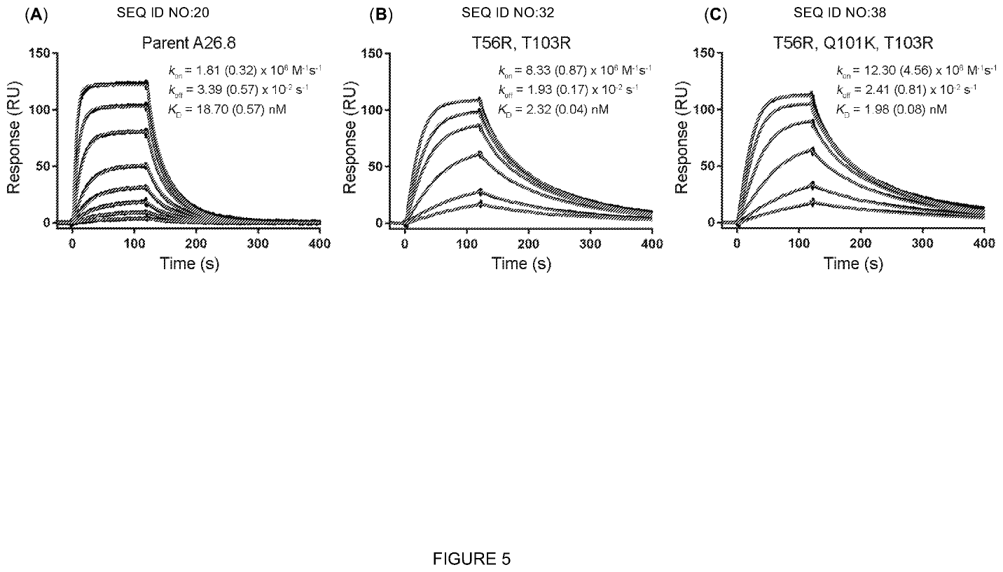

The antigen binding kinetic and affinity constant data determined by SPR measurements for multiple-point mutants are presented in TABLE 3 and FIG. 3. All 10 multiple-mutants show improved binding affinities relative to the parent A26.8 V_{H}H antibody, and 8 of them also show improvements relative to the best single-point mutant, T103R. The best double mutant is T56R,T103R combining the two best single mutations, which occur in CDR2 and CDR3. The 8-fold affinity improvement comes from an over 6-fold increase in kₒₙ coupled with a small but statistically significant decrease in k_{off}, relative to the parent A26.8 V_{H}H. The improvement in affinity does not come at the cost of stability. The best mutant tested (T56R,Q101K,T103R) is a triple mutant that incorporates the beneficial Q101K mutation into the best double mutant. This leads to an overall 10-fold improvement of K_{D}, with an ˜7-fold effect attributed to the change in kₒₙ, as well as a 3.4° C. increase in Tₘ relative to the original A26.8. Not only are the best multiple mutants composed of the best single mutants, but there is also a generally robust additivity of mutation effects throughout the entire data set (FIG. 4). Examples of SPR sensorgrams are shown in the FIG. 5 for the best double and triple mutants comparatively to those of the parent molecule, visually illustrating changes in the kinetic behavior that are favorable to the mutants.

### EXAMPLE 4

### Thermal Stability Measurements

Differential scanning fluorimetry (DSF) was used to determine the melting temperatures (Tₘs) of the parental and mutant A26.8 variants. DSF was carried out in a Rotor-Gene 6000 real-time PCR instrument (Corbett Life Science, Mortlake, NSW, Australia). Samples were diluted in HyClone™ Dulbecco's phosphate-buffered saline (D-PBS; GE Healthcare) at a final concentration, after mixing, of 0.33 mg/mL. A total volume of 30 μL in 0.2 mL thin wall PCR tubes (Axygen, Oneonta, N.Y.) was used. SYPRO® Orange (Life Technologies, Burlington, ON, Canada) was diluted 1,000-fold from the 5,000× concentrated stock to the working dye solution in D-PBS and 15 μL were added to 15 μL of sample just prior to the experiment. Thermal denaturation was carried out by increasing the temperature from 30° C. to 94° C. at a rate of 0.06° C./s. Fluorescence intensity, with excitation at 470 nm and emission at 610 nm, was collected at 1° C. intervals and analyzed with the Rotor Gene 6000 series software v1.7 (Corbett Life Science). The Tₘ values were determined from the peak of the first derivative transformation of the raw data. Each sample was measured in quadruplicate.

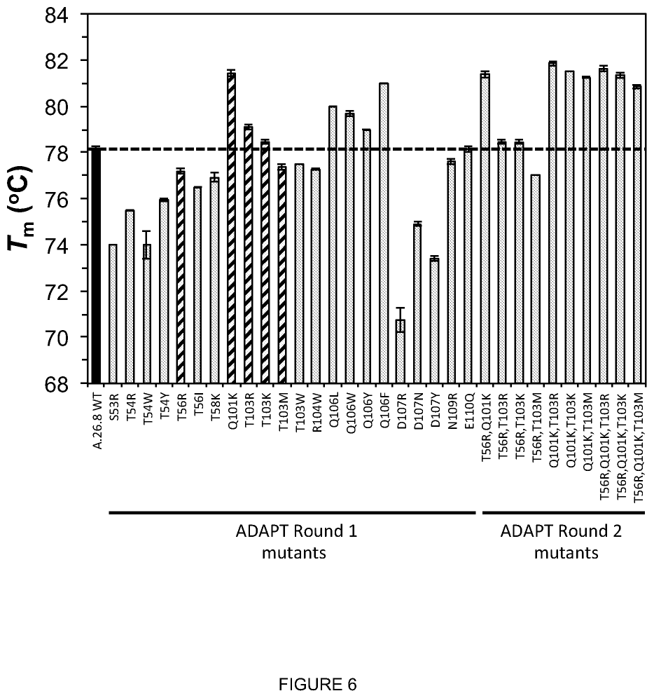

Most of the first-round single-mutants had thermal stabilities similar to the parental A26.8 sdAb with a Tₘ of about 78° C. (TABLE 3). This was expected since the FoldX stability scores were used to filter out mutations predicted to be significantly destabilizing. Mutations Q101K, T103R and those at position 106 had a stabilizing effect relative to the wild-type (FIG. 6). The most destabilizing effect was observed for the D107R mutant; however its Tₘ still remained above 70° C.

Determination of the Tₘs for the 10 double- and triple-mutants indicate that 7 have increased stabilities, 2 have similar stabilities and one has a slightly decreased stability relative to parental A26.8 (TABLE 3, FIG. 5). The increase in thermal stability can be associated with the presence of the Q101K mutation, consistent with the ˜3° C. increase in Tₘ seen for this single-point mutant.

### EXAMPLE 5

### Toxin A Inhibition Assay

Vero cells (CCL-81; ATCC, Manassas, Va.) were maintained in complete media (MEM+antibiotic/antimycotic+10% FBS) in T-75 flasks at 37° C., 5% CO₂ For TcdA inhibition assays, sterile 96-well tissue culture plates (Thermo-Fisher, Ottawa, ON, Canada) were seeded with ˜2×10⁴ cells/well in a total volume of 200 μL of complete media. Plates were incubated for 24 h at 37° C., 5% CO₂. Media was then carefully removed from each well and replaced with 200 μL of fresh media, 20 μL of antibody (serially diluted in sterile PBS, giving final in-well concentrations ranging from 1 μM to 1.95 nM) and 10 μL of TcdA (List Biological Laboratories; final in-well concentration of 10 ng/mL) for all wells. Control wells received 20 μL of PBS instead of antibody, or 10 μL of PBS instead of TcdA. Plates were then incubated at 37° C. and 5% CO₂ for 72 h. Next, the media was removed and replaced with 100 μL of pre-warmed MEM containing 10% WST-1 cell proliferation reagent (Roche, Laval, QC, Canada). Plates were incubated at 37° C. and 5% CO₂ for 1 h before reading the absorbance at 450 nm using a Multiskan™ FC photometer (Thermo-Fisher). Data were analyzed for % inhibition of TcdA (relative to untreated control wells) and graphed using GraphPad Prism software. The reported values are mean TcdA inhibition derived from four independent assays.

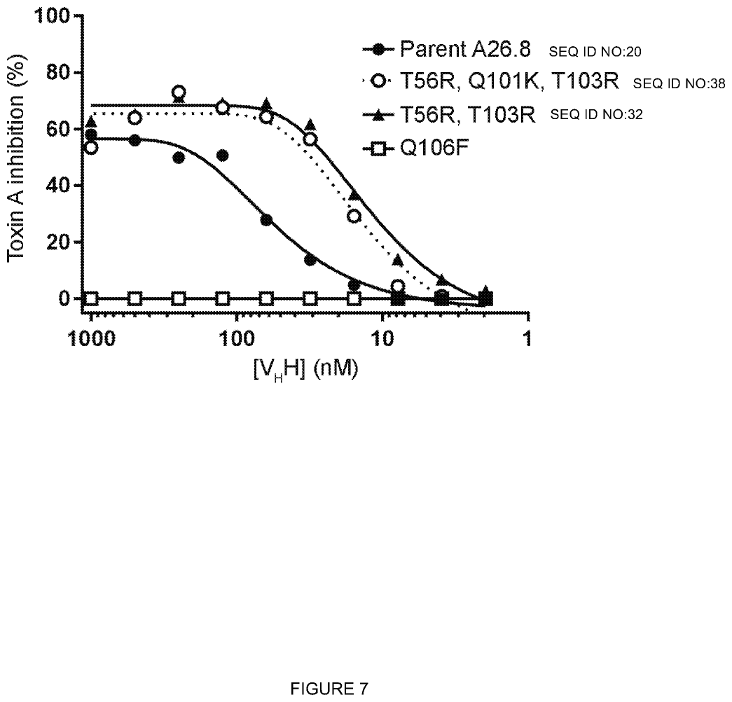

The antigen-binding affinity improvements obtained at the end of the second cycle of ADAPT prompted us to test the best binders for TcdA neutralization at the cellular level. TcdA-induced Vero cell cytotoxicity data as a function of V_{H}H concentration are shown in FIG. 7. It is clear that the best affinity-matured variants (double-mutant T56R,T103R and triple-mutant T56R,Q101K,T103R) have an increased protective effect against C. difficile TcdA relative to the parent V_{H}H. In this assay, both mutant variants show about the same TcdA IC₅₀ of ˜12 nM versus ˜70 nM for the parent A26.8 V_{H}H, a 6-fold increase in neutralization potency. It is also interesting to note that the affinity-matured variants can reach maximal TcdA inhibition levels around 70% (at 60 nM V_{H}H), whereas the parent V_{H}H can only reach ˜60% and that at a much higher concentration (500 nM). As a negative control, we also tested one of the single-point mutants with reduced antigen binding affinity, Q106F, which as expected failed to inhibit TcdA cytotoxicity (FIG. 7). Taken together, these data demonstrate that ADAPT-guided optimization of the A26.8 V_{H}H paratope for improved antigen-binding affinity can translate into a marked enhancement of TcdA neutralization at the cellular level.

### EXAMPLE 6

### Structural Interpretation of Enhanced Affinity and Stability

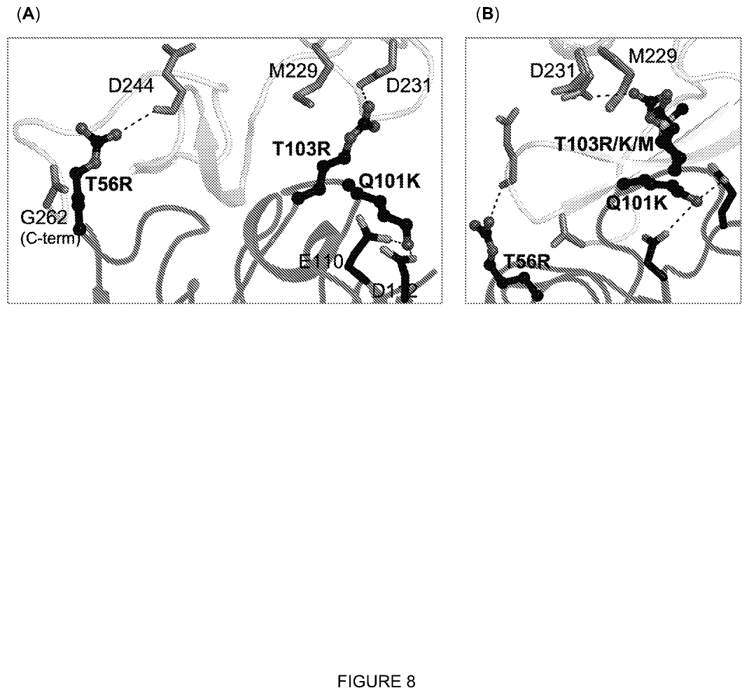

One of the advantages of rational structure-guided affinity maturation is that it helps to understand the structural basis for improvement of binding affinity. In FIG. 8A we display the atomic-level interactions predicted for the triple mutant, T56R,Q101K,T103R, with the TcdA fragment. It can be seen that both of the main contributors to the affinity improvement, the arginines at positions 56 and 103, establish novel, relatively short-range electrostatic interactions with negatively charged groups of the antigen (D231, D244, and G262 C-terminus). The lysine at the more peripheral position 101 seems to act by neutralizing some of the negative charges on the V_{H}H itself (at E110 and D112 of CDR3) hence alleviating some of the long-range electrostatic repulsion to the antigen present in the parental molecule. The new intramolecular H-bond interactions introduced by Lys at position 101 (FIG. 8) are also likely responsible for the observed increase in stability upon the Q101K mutation (TABLE 3, FIG. 5). The aliphatic portion of R103 may also benefit from novel short-range contacts with M229 of the antigen. As shown in the rotated view of FIG. 8B, the hydrophobic Met side chain at position 103 could still contact M229 of the antigen, but lacks the potential of Arg or Lys for electrostatic interactions, thereby leading to a smaller improvement in binding affinity (FIG. 3, TABLE 3). Between the two positively charged side-chain substitutions at position 103, Arg is favored according to the SPR measurements, in agreement with structural predictions showing that the shorter Lys side chain does not reach close enough to D231 of the antigen in order to establish an H-bonding interaction as in the case of R103 (FIG. 8B).

### REFERENCES

All patents, patent applications and publications referred to herein and throughout the application are hereby incorporated by reference.

Arbabi-Ghahroudi, M. et al. (2009) Protein Eng. Des. Sel. 22, 59-66.

Babcock, G. J., Broering, T. J., Hernandez, H. J., Mandell, R. B., Donahue, K., Boatright, N., Stack, A. M., Lowy, I., Graziano, R., Molrine, D., Ambrosino, D. M., and Thomas, W. D. Jr. (2006) Infect. Immun. 74, 6339-6347.

Bell, A., Wang, Z. J., Arbabi-Ghahroudi, M., Chang, T. A., Durocher, Y., Trojahn, U., Baardsnes, J., Jaramillo, M. L., Li, S., Baral, T. N., O'Connor-McCourt, M., Mackenzie, R., and Zhang, J. (2010) Cancer Lett. 289, 81-90.

Chothia, C., and Lesk, A. M. (1987) J. Mol. Biol. 196, 901-917.

Cornell, W. D. et al. (1995) J. Am. Chem. Soc. 117, 5179-5197.

Corthier, G., Muller, M. C., Wilkins, T. D., Lyerly, D., and L'Haridon, R. (1991) Infect. Immun. 59, 1192-1195.

De Kruif, J., and Logtenberg, T. (1996) J. Biol. Chem. 271, 7630-7634.

Eisenberg, D., Schwarz, E., Komaromy, M., and Wall, R. (1984) J. Mol. Biol. 179, 125-142

Fenner, L., Widmer, A. F., Goy, G., Rudin, S., and Frei, R. (2008) J. Clin. Microbiol. 46, 328-330.

Gardiner, D. F., Rosenberg, T., Zaharatos, J., Franco, D., and Ho, D. D. (2009) Vaccine 27, 3598-3604.

Giannasca, P. J., Zhang, Z. X., Lei, W. D., Boden, J. A., Giel, M. A., Monath, T. P., and Thomas, W. D. Jr. (1999) Infect. Immun. 67, 527-538.

Guerois, R., Nielsen, J. E. and Serrano, L. (2002) J. Mol. Biol. 320, 369-387.

Hamers-Casterman, C., Atarhouch, T., Muyldermans, S., Robinson, G., Hamers, C., Songa, E. B., Bendahman, N., and Hamers, R. (1993) Nature 363, 446-448.

Hassoun, A., and Ibrahim, F. (2007) Am. J. Geriatr. Pharmacother. 5, 48-51.

Hornak, V. et al. (2006) Proteins 65, 712-725.

Hussack, G. et al. (2011) J. Biol. Chem. 286, 8961-8976.

Iqbal, U., Trojahn, U., Albaghdadi, H., Zhang, J., O'Connor, M., Stanimirovic, D., Tomanek, B., Sutherland, G., and Abulrob, A. (2010) Br. J. Pharmacol. 160, 1016-1028.

Jank, T., and Aktories, K. (2008) Trends Microbiol. 16, 222-229.

Jank, T., Giesemann, T., and Aktories, K. (2007) Glycobiology 17, 15R-22R.

Jespers, L., Schon, O., Famm, K., and Winter, G. (2004) Nat. Biotechnol. 22, 1161-1165.

Johal, S. S., Lambert, C. P., Hammond, J., James, P. D., Borriello, S. P., and Mahida, Y. R. (2004) J. Clin. Pathol. 57, 973-979.

Johnson, S. (2009) J. Infect. 58, 403-410.

Juang, P., Skledar, S. J., Zgheib, N. K., Paterson, D. L., Vergis, E. N., Shannon, W. D., Ansani, N. T., and Branch, R. A. (2007) Am. J. Infect. Control 35, 131-137.

Kabat, E. A., and Wu, T. T. (1991) J. Immunol. 147:1709-19.

Katchar, K., Taylor, C. P., Tummala, S., Chen, X., Sheikh, J., and Kelly, C. P. (2007) Clin. Gastroenterol. Hepatol. 5, 707-713.

Kelly, C. P., Chetham, S., Keates, S., Bostwick, E. F., Roush, A. M., Castagliuolo, I., LaMont J. T., and Pothoulakis, C. (1997) Antimicrob. Agents Chemother. 41, 236-241.

Kelly, C. P., Pothoulakis, C., and LaMount, J. T. (1994) N. Engl. J. Med. 330, 257-262.

Kelly, C. P., Pothoulakis, C., Orellana, J., and LaMont, J. T. (1992) Gastroenterology 102, 35-40 (2009) Nat. Rev. Drug Discov. 8, 442.

Kelly, C. P., Pothoulakis, C., Vavva, F., Castagliuolo, I., Bostwick, E. F., O'Keane, J. C., Keates, S., and LaMont, J. T. (1996) Antimicrob. Agents Chemother. 40, 373-379.

Kink, J. A., and Williams, J. A. (1998) Infect. Immun. 66, 2018-2025.

Krivov, G. G., Shapovalov, M. V. and Dunbrack, R. L., Jr. (2009) Proteins 77, 778-795.

Kyne, L., Hamel, M. B., Polavaram, R., and Kelly, C. P. (2002) Clin. Infect. Dis. 34, 346-353.

Kyne, L., Warny, M., Qamar, A., and Kelly, C. P. (2000) N. Engl. J. Med. 340, 390-397.

Kyne, L., Warny, M., Qamar, A., and Kelly, C. P. (2001) Lancet 357, 189-193.

Leffler, D. A., and Lamont, J. T. (2009) Gastroenterology 136, 1899-1912.

Lefranc, M.-P. et al.,(2003) Dev. Comp. Immunol., 27, 55-77.

Leung, D. Y., Kelly, C. P., Boguniewicz, M., Pothoulakis, C., LaMont, J. T., and Flores, A. (1991) J. Pediatr. 118, 633-637.

Lowy, I., Molrine, D. C., Leav, B. A., Blair, B. M., Baxter, R., Gerding, D. N., Nichol, G., Thomas, W. D. Jr., Leney, M., Sloan, S., Hay, C. A., and Ambrosino, D. M. (2010) N. Engl. J. Med. 362, 197-205.

Lyerly, D. M., Bostwick, E. F., Binion, S. B., and Wilkins, T. D. (1991) Infect. Immun. 59, 2215-2218.

Lyras, D., O'Connor, J. R., Howarth, P. M., Sambol, S. P., Carter, G. P., Phumoonna, T., Poon, R., Adams, V., Vedantam, G., Johnson, S., Gerding, D. N., and Rood, J. I. (2009) Nature 458, 1176-1179.

McPherson, S., Rees, C. J., Ellis, R., Soo, S., and Panter, S. J. (2006) Dis. Colon Rectum 49, 640-645.

Murase, T. et al. (2014) J. Biol. Chem. 289, 2331-2343.

Merritt, E. A., and Hol, W. G. (1995) Curr. Opin. Struct. Biol. 5, 165-171.

Musher, D. M., Manhas, A., Jain, P., Nuila, F., Waqar, A., Logan, N., Marino, B., Graviss, E. A. (2007) J. Clin. Microbiol. 45, 2737-2739.

Naim, M. et al. (2007) J. Chem. Inf. Model. 47, 122-133.

Nuttall, S. D., Krishnan, U. V., Doughty, L., Pearson, K., Ryan, M. T., Hoogenraad, N. J., Hattarki, M., Carmichael, J. A., Irving, R. A., and Hudson, P. J. (2003) Eur. J. Biochem. 270, 3543-3554.

O'Conchuir, S. et al. (2015) PLoS One 10, e0130433.

O'Connor, J. R., Johnson, S., and Gerding, D. N. (2009) Gastroenterology 136, 1913-1924.

Pépin, J., Valiquette, L., and Cossette, B. (2005) CMAJ 173, 1037-1042.

Planche, T., Aghaizu, A., Holliman, R., Riley, P., Poloniecki, J., Breathnach, A., and Krishna, S. (2008) Lancet Infect. Dis. 8, 777-784.

Vivcharuk. et al. (2017) PLoS One, in press.

Ridgway, J. B., Presta, L. G., and Carter, P. (1996) Protein Eng. 9, 617-621.

Rohl, C. A., Strauss, C. E., Misura, K. M. and Baker, D. (2004) Methods Enzymol. 383, 66-93.

Rupnik, M., Wilcox, M. H., and Gerding, D. N. (2009) Nat. Rev. Microbiol. 7, 526-536.

Rüssmann, H., Panthel, K., Bader, R. C., Schmitt, C., and Schaumann, R. (2007) Eur. J. Clin. Microbiol. Infect. Dis. 26, 115-119.

Salcedo, J., Keates, S., Pothoulakis, C., Warny, M., Castagliuolo, I., LaMont, J. T., and Kelly, C. P. (1997) Gut 41, 366-370.

Schymkowitz, J. et al. (2005) Nucl. Acids Res. 33, W382-388.

Sloan, L. M., Duresko, B. J., Gustafson, D. R., and Rosenblatt, J. E. (2008) J. Clin. Microbiol. 46, 1996-2001.

Songer, J. G. (2004) Anim. Health Res. Rev. 5, 321-326.

Sulea, T. and Purisima, E. O. (2012) Methods Mol. Biol. 819, 295-303.

Sulea, T., Vivcharuk, V., Corbeil, C. R., Deprez, C. and Purisima, E. O. (2016) J. Chem. Inf. Model. 56, 1292-1303.

Tjellström, B., Stenhammar, L., Eriksson, S., and Magnusson, K. E. (1993) Lancet 341, 701-702.

Turgeon, D. K., Novicki, T. J., Quick, J., Carlson, L., Miller, P., Ulness, B., Cent, A., Ashley, R., Larson, A., Coyle, M., Limaye, A. P., Cookson, B. T., and Fritsche, T. R. (2003) J. Clin. Microbiol. 41, 667-670.

Viscidi, R., Laughon, B. E., Yolken, R., Bo-Linn, P., Moench, T., Ryeder, R. W., and Bartlett, J. G. (1983) J. Infect. Dis. 148, 93-100.

Warny, M., Fatimi, A., Bostwick, E. F., Laine, D. C., Label, F., LaMont, J. T., Pothoulakis, C., and Kelly, C. P. (1999) Gut 44, 212-217.

Warny, M., Pepin, J., Fang, A., Killgore, G., Thompson, A., Brazier, J., Frost, E., and McDonald, L. C. (2005) Lancet 366, 1079-1084.

Warny, M., Vaerman, J. P., Avesani, V., and Delmée, M. (1994) Infect. Immun. 62, 384-389.

Wesolowski, J., Alzogaray, V., Reyelt, J., Unger, M., Juarez, K., Urrutia, M., Cauerhff, A., Danguah, W., Rissiek, B., Scheuplein, F., Schwarz, N., Adriouch, S., Boyer, O., Seman, M., Licea, A., Serreze, D. V., Goldbaum, F. A., Haag, F., and Koch-Nolte, F. (2009) Med. Microbiol. Immunol. 198, 157-174.

Wilcox, N. H. (2004) J. Antimicrob. Chemother. 53, 882-884.

Wilkins, T. D., and Lyerly, D. M. (2003) J. Clin. Microbiol. 41, 531-534.

Zhang, J., Li, Q., Nguyen, T.-D., Tremblay, T.-L., Stone, E., To, R., Kelly, J., and MacKenzie, C. R. (2004a) J. Mol. Biol. 341, 161-169.

Zhang, J., Tanha, J., Hirama, T., Khiew, N. H., To, R., Tong-Sevinc, H., Stone, E., Brisson, J. R., and MacKenzie, C. R. (2004b) J. Mol. Biol. 335, 49-56.

TABLE 4
CDR sequence listing of A26.8 variants.

| | | | | | | Corresponding | |
|---|---|---|---|---|---|---|---|
| CDR1 | | CDR2 | | CDR3 | | A26.8 variant | |
| | SEQ | | SEQ | | SEQ | | SEQ |
| | ID | | ID | | ID | | ID |
| Sequence | NO | Sequence | NO | Sequence | NO | Name | NO |
| ERTFSRYP | 1 | ISSX₁GX₂SX₃ | 2 | AVNSX₄RX₅RLQDPX₆EYDY | 3 | A26.8-generic | 19 |
| ERTFSRYP | 1 | ISSTGTST | 4 | AVNSQRTRLQDPNEYDY | 5 | A26.8-wild-type | 20 |
| ERTFSRYP | 1 | ISSRGTST | 6 | AVNSQRTRLQDPNEYDY | 5 | A26.8-T54R | 21 |
| ERTFSRYP | 1 | ISSTGRST | 7 | AVNSQRTRLQDPNEYDY | 5 | A26.8-T56R | 22 |
| ERTFSRYP | 1 | ISSTGIST | 8 | AVNSQRTRLQDPNEYDY | 5 | A26.8-T56I | 23 |
| ERTFSRYP | 1 | ISSTGTSK | 9 | AVNSQRTRLQDPNEYDY | 5 | A26.8-T58K | 24 |
| ERTFSRYP | 1 | ISSTGTST | 4 | AVNSKRTRLQDPNEYDY | 10 | A26.8-Q101K | 25 |
| ERTFSRYP | 1 | ISSTGTST | 4 | AVNSQRRRLQDPNEYDY | 11 | A26.8-T103R | 26 |
| ERTFSRYP | 1 | ISSTGTST | 4 | AVNSQRKRLQDPNEYDY | 12 | A26.8-T103K | 27 |
| ERTFSRYP | 1 | ISSTGTST | 4 | AVNSQRMRLQDPNEYDY | 13 | A26.8-T103M | 28 |
| ERTFSRYP | 1 | ISSTGTST | 4 | AVNSQRWRLQDPNEYDY | 14 | A26.8-T103W | 29 |
| ERTFSRYP | 1 | ISSTGTST | 4 | AVNSQRTRLQDPREYDY | 15 | A26.8-N109R | 30 |
| ERTFSRYP | 1 | ISSRGTST | 6 | AVNSKRTRLQDPNEYDY | 10 | A26.8-T56R, Q101K | 31 |
| ERTFSRYP | 1 | ISSRGTST | 6 | AVNSQRRRLQDPNEYDY | 11 | A26.8-T56R, T103R | 32 |
| ERTFSRYP | 1 | ISSRGTST | 6 | AVNSQRKRLQDPNEYDY | 12 | A26.8-T56R, T103K | 33 |
| ERTFSRYP | 1 | ISSRGTST | 6 | AVNSQRMRLQDPNEYDY | 13 | A26.8-T56R, T103M | 34 |
| ERTFSRYP | 1 | ISSTGTST | 4 | AVNSKRRRLQDPNEYDY | 16 | A26.8-Q101K,T103R | 35 |
| ERTFSRYP | 1 | ISSTGTST | 4 | AVNSKRKRLQDPNEYDY | 17 | A26.8-Q101K,T103K | 36 |
| ERTFSRYP | 1 | ISSTGTST | 4 | AVNSKRMRLQDPNEYDY | 18 | A26.8-Q101K,T103M | 37 |
| ERTFSRYP | 1 | ISSRGTST | 6 | AVNSKRRRLQDPNEYDY | 16 | A26.8-T56R, Q101K, T103R | 38 |
| ERTFSRYP | 1 | ISSRGTST | 6 | AVNSKRKRLQDPNEYDY | 17 | A26.8-T56R, Q101K, T103K | 39 |
| ERTFSRYP | 1 | ISSRGTST | 6 | AVNSKRMRLQDPNEYDY | 18 | A26.8-T56R, Q101K, T103M | 40 |

TABLE 5
Complete sequence listing of A26.8 variants.

| | SEQ | |
|---|---|---|
| | ID | |
| Name A28.8 variant | NO | Sequence (CDR sequences are [symbol] highlighted in bold) |
| A26.8-generic | 19 | QVKLEESGGGLVQAGGSLRLSCAASERTFSRYPVAWFRQAPGAEREFVAVISSX₁GX₂SX₃WADSV |
| | | KGRFTISRDNAKVTVYLQMNNLKREDTAVYFCAVNSX₄RX₅RLQDPX₆EYDYVVGQGTQVTVSS |
| A26.8-wild-type | 20 | QVKLEESGGGLVQAGGSLRLSCAASERTFSRYPVAWFRQAPGAEREFVAVISSTGTSTYYADSVKG |
| | | RFTISRDNAKVTVYLQMNNLKREDTAVYFCAVNSQRTRLQDPNEYDYWGQGTQVTVSS |
| A26.8-T54R | 21 | QVKLEESGGGLVQAGGSLRLSCAASERTFSRYPVAWFRQAPGAEREFVAVISSRGTSTYYADSVK |
| | | GRFTISRDNAKVTVYLQMNNLKREDTAVYFCAVNSQRTRLQDPNEYDYWGQGTQVTVSS |
| A26.8-T56R | 22 | QVKLEESGGGLVQAGGSLRLSCAASERTFSRYPVAWFRQAPGAEREFVAVISSTGRSTYYADSVK |
| | | GRFTISRDNAKVTVYLQMNNLKREDTAVYFCAVNSQRTRLQDPNEYDYWGQGTQVTVSS |
| A26.8-T56I | 23 | QVKLEESGGGLVQAGGSLRLSCAASERTFSRYPVAWFRQAPGAEREFVAVISSTGISTYYADSVKG |
| | | RFTISRDNAKVTVYLQMNNLKREDTAVYFCAVNSQRTRLQDPNEYDYWGQGTQVTVSS |
| A26.8-T58K | 24 | QVKLEESGGGLVQAGGSLRLSCAASERTFSRYPVAWFRQAPGAEREFVAVISSTGKSTYYADSVK |
| | | GRFTISRDNAKVTVYLQMNNLKREDTAVYFCAVNSQRTRLQDPNEYDYWGQGTQVTVSS |
| A26.8-Q101K | 25 | QVKLEESGGGLVQAGGSLRLSCAASERTFSRYPVAWFRQAPGAEREFVAVISSTGTSTYYADSVKG |
| | | RFTISRDNAKVTVYLQMNNLKREDTAVYFCAVNSKRTRLQDPNEYDYWGQGTQVTVSS |
| A26.8-T103R | 26 | QVKLEESGGGLVQAGGSLRLSCAASERTFSRYPVAWFRQAPGAEREFVAVISSTGTSTYYADSVKG |
| | | RFTISRDNAKVTVYLQMNNLKREDTAVYFCAVNSQRRRLQDPNEYDYWGQGTQVTVSS |
| A26.8-T103K | 27 | QVKLEESGGGLVQAGGSLRLSCAASERTFSRYPVAWFRQAPGAEREFVAVISSTGTSTYYADSVKG |
| | | RFTISRDNAKVTVYLQMNNLKREDTAVYFCAVNSQRKRLQDPNEYDYWGQGTQVTVSS |
| A26.8-T103M | 28 | QVKLEESGGGLVQAGGSLRLSCAASERTFSRYPVAWFRQAPGAEREFVAVISSTGTSTYYADSVKG |
| | | RFTISRDNAKVIVYLQMNNLKREDTAVYFCAVNSQRMRLQDPNEYDYVVGQGTQVTVSS |
| A26.8-T103W | 29 | QVKLEESGGGLVQAGGSLRLSCAASERTFSRYPVAWFRQAPGAEREFVAVISSTGTSTYYADSVKG |
| | | RFTISRDNAKVTVYLQMNNLKREDTAVYFCAVNSQRWRLQDPNEYDYWGQGTQVTVSS |
| A26.8-N109R | 30 | QVKLEESGGGLVQAGGSLRLSCAASERTFSRYPVAWFRQAPGAEREFVAVISSTGTSTYYADSVKG |
| | | RFTISRDNAKVTVYLQMNNLKREDTAVYFCAVNSQRTRLQDPREYDYWGQGTQVTVSS |
| A26.8-156R, Q101K | 31 | QVKLEESGGGLVQAGGSLRLSCAASERTFSRYPVAWFRQAPGAEREFVAVISSTGRSTYYADSVK |
| | | GRFTISRDNAKVTVYLQMNNLKREDTAVYFCAVNSKRTRLQDPNEYDYVVGQGTQVTVSS |
| A26.8-156R, T103R | 32 | QVKLEESGGGLVQAGGSLRLSCAASERTFSRYPVAWFRQAPGAEREFVAVISSTGRSTYYADSVK |
| | | GRFTISRDNAKVIVYLQMNNLKREDTAVYFCAVNSQRRRLQDPNEYDYVVGQGTQVTVSS |
| A26.8-156R, T103K | 33 | QVKLEESGGGLVQAGGSLRLSCAASERTFSRYPVAWFRQAPGAEREFVAVISSTGRSTYYADSVK |
| | | GRFTISRDNAKVIVYLQMNNLKREDTAVYFCAVNSQRKRLQDPNEYDYVVGQGTQVIVSS |
| A26.8-156R, T103M | 34 | QVKLEESGGGLVQAGGSLRLSCAASERTFSRYPVAWFRQAPGAEREFVAVISSTGRSTYYADSVK |
| | | GRFTISRDNAKVIVYLQMNNLKREDTAVYFCAVNSQRMRLQDPNEYDYVVGQGTQVTVSS |
| A26.8-Q101K, T103R | 35 | QVKLEESGGGLVQAGGSLRLSCAASERTFSRYPVAWFRQAPGAEREFVAVISSTGTSTYYADSVKG |
| | | RFTISRDNAKVIVYLQMNNLKREDTAVYFCAVNSKRRRLQDPNEYDYWGQGTQVTVSS |
| A26.8-Q101K, T103K | 36 | QVKLEESGGGLVQAGGSLRLSCAASERTFSRYPVAWFRQAPGAEREFVAVISSTGTSTYYADSVKG |
| | | RFTISRDNAKVTVYLQMNNLKREDTAVYFCAVNSKRKRLQDPNEYDYWGQGTQVTVSS |
| A26.8-Q101K, T103M | 37 | QVKLEESGGGLVQAGGSLRLSCAASERTFSRYPVAWFRQAPGAEREFVAVISSTGTSTYYADSVKG |
| | | RFTISRDNAKVTVYLQMNNLKREDTAVYFCAVNSKRMRLQDPNEYDYWGQGTQVTVSS |
| A26.8- | 38 | QVKLEESGGGLVQAGGSLRLSCAASERTFSRYPVAWFRQAPGAEREFVAVISSTGRSTYYADSVK |
| 156R, Q101K, T103R | | GRFTISRDNAKVTVYLQMNNLKREDTAVYFCAVNSKRRRLQDPNEYDYWGQGTQVTVSS |
| A26.8- | 39 | QVKLEESGGGLVQAGGSLRLSCAASERTFSRYPVAWFRQAPGAEREFVAVISSTGRSTYYADSVK |
| 156R, Q101K, T103K | | GRFTISRDNAKVTVYLQMNNLKREDTAVYFCAVNSKRKRLQDPNEYDYWGQGTQVTVSS |
| A26.8- | 40 | QVKLEESGGGLVQAGGSLRLSCAASERTFSRYPVAWFRQAPGAEREFVAVISSTGRSTYYADSVK |
| 156R, Q101K, T103M | | GRFTISRDNAKVTVYLQMNNLKREDTAVYFCAVNSKRMRLQDPNEYDYWGQGTQVTVSS |

1. An isolated, purified or recombinant antibody or fragment comprising a sequence of CDR1 of ERTFSRYP (SEQ ID NO:1); CDR2 of ISSX₁GX₂SX₃ (SEQ ID NO:2); and CDR3 of AVNSX₄RX₅RLQDPX₆EYDY (SEQ ID NO: 3);
wherein: X₁ is T or R; X₂ is T, R or I; X₃ is T or K; X₄ is Q or K; X₅ is T, R, K, M or W; and X₆ is N or R; and combinations thereof; and
with the proviso that when CDR2 is ISSTGTST (SEQ ID NO:4) then CDR3 is not AVNSQRTRLQDPNEYDY (SEQ ID NO: 5), and vice-versa; and
wherein the antibody or fragment thereof is specific to TcdA.

2. The antibody or fragment of claim 1 which binds to TcdA with greater affinity than an antibody or fragment comprising CDR1 of SEQ ID NO:1, CDR2 of SEQ ID NO:4, and CDR3 of SEQ ID NO:5.

3. An isolated, purified or recombinant antibody or fragment of claim 1 selected from the group consisting of:
an isolated, purified or recombinant antibody or fragment thereof comprising a sequence of CDR1 of SEQ ID NO:1; CDR2 of SEQ ID NO:6; and CDR3 of SEQ ID NO: 5;
an isolated, purified or recombinant antibody or fragment thereof comprising a sequence of CDR1 of SEQ ID NO:1; CDR2 of SEQ ID NO:7; and CDR3 of SEQ ID NO: 5;
an isolated, purified or recombinant antibody or fragment thereof comprising a sequence of CDR1 of SEQ ID NO:1; CDR2 of SEQ ID NO:8; and CDR3 of SEQ ID NO: 5;
an isolated, purified or recombinant antibody or fragment thereof comprising a sequence of CDR1 of SEQ ID NO:1; CDR2 of SEQ ID NO:9; and CDR3 of SEQ ID NO: 5;
an isolated, purified or recombinant antibody or fragment thereof comprising a sequence of CDR1 of SEQ ID NO:1; CDR2 of SEQ ID NO:4; and CDR3 of SEQ ID NO: 10;
an isolated, purified or recombinant antibody or fragment thereof comprising a sequence of CDR1 of SEQ ID NO:1; CDR2 of SEQ ID NO:4; and CDR3 of SEQ ID NO: 11;
an isolated, purified or recombinant antibody or fragment thereof comprising a sequence of CDR1 of SEQ ID NO:1; CDR2 of SEQ ID NO:4; and CDR3 of SEQ ID NO: 12;
an isolated, purified or recombinant antibody or fragment thereof comprising a sequence of CDR1 of SEQ ID NO:1; CDR2 of SEQ ID NO:4; and CDR3 of SEQ ID NO: 13;
an isolated, purified or recombinant antibody or fragment thereof comprising a sequence of CDR1 of SEQ ID NO:1; CDR2 of SEQ ID NO:4; and CDR3 of SEQ ID NO: 14;
an isolated, purified or recombinant antibody or fragment thereof comprising a sequence of CDR1 of SEQ ID NO:1; CDR2 of SEQ ID NO:4; and CDR3 of SEQ ID NO: 15;
an isolated, purified or recombinant antibody or fragment thereof comprising a sequence of CDR1 of SEQ ID NO:1; CDR2 of SEQ ID NO:6; and CDR3 of SEQ ID NO: 10;
an isolated, purified or recombinant antibody or fragment thereof comprising a sequence of CDR1 of SEQ ID NO:1; CDR2 of SEQ ID NO:6; and CDR3 of SEQ ID NO: 11;
an isolated, purified or recombinant antibody or fragment thereof comprising a sequence of CDR1 of SEQ ID NO:1; CDR2 of SEQ ID NO:6; and CDR3 of SEQ ID NO: 12;
an isolated, purified or recombinant antibody or fragment thereof comprising a sequence of CDR1 of SEQ ID NO:1; CDR2 of SEQ ID NO:6; and CDR3 of SEQ ID NO: 13;
an isolated, purified or recombinant antibody or fragment thereof comprising a sequence of CDR1 of SEQ ID NO:1; CDR2 of SEQ ID NO:4; and CDR3 of SEQ ID NO: 16;
an isolated, purified or recombinant antibody or fragment thereof comprising a sequence of CDR1 of SEQ ID NO:1; CDR2 of SEQ ID NO:4; and CDR3 of SEQ ID NO: 17;
an isolated, purified or recombinant antibody or fragment thereof comprising a sequence of CDR1 of SEQ ID NO:1; CDR2 of SEQ ID NO:4; and CDR3 of SEQ ID NO: 18;
an isolated, purified or recombinant antibody or fragment thereof comprising a sequence of CDR1 of SEQ ID NO:1; CDR2 of SEQ ID NO:6; and CDR3 of SEQ ID NO: 16;
an isolated, purified or recombinant antibody or fragment thereof comprising a sequence of CDR1 of SEQ ID NO:1; CDR2 of SEQ ID NO:6; and CDR3 of SEQ ID NO: 17;
an isolated, purified or recombinant antibody or fragment thereof comprising a sequence of CDR1 of SEQ ID NO:1; CDR2 of SEQ ID NO:6; and CDR3 of SEQ ID NO: 18.

4. An isolated, purified or recombinant antibody or fragment of claim 3 selected from the group consisting of:
(SEQ ID NO: 19) 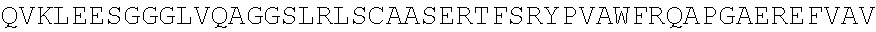 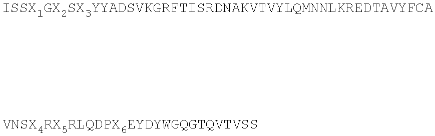
wherein: X₁ is T or R; X₂ is T, R or I; X₃ is T or K; X₄ is Q or K; X₅ is T, R, K, M or W; and X₆ is N or R; and combinations thereof;
and excluding SEQ ID NO: 20.

5. An isolated, purified or recombinant antibody or fragment of claim 4 selected from the group consisting of:
(SEQ ID NO: 21) 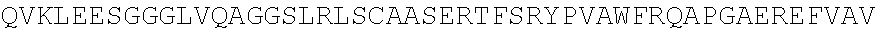 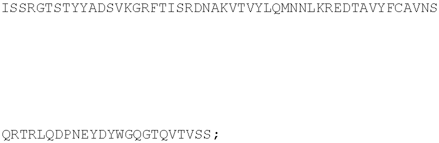
(SEQ ID NO: 22) 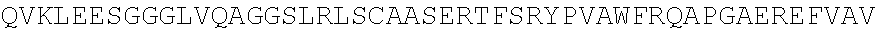 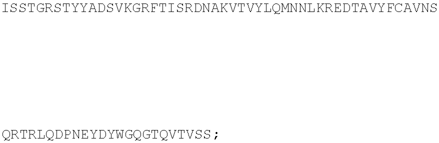
(SEQ ID NO: 23) 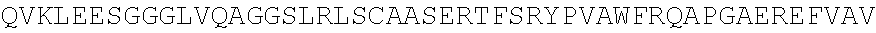 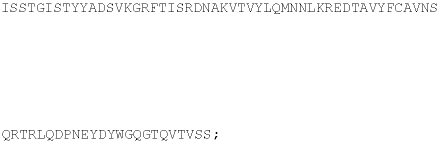
(SEQ ID NO: 24) 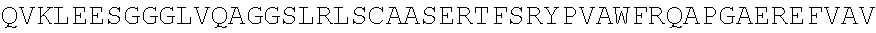 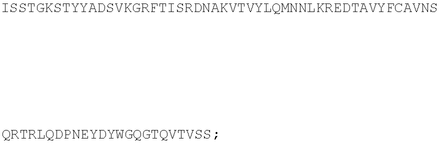
(SEQ ID NO: 25) 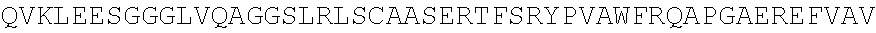 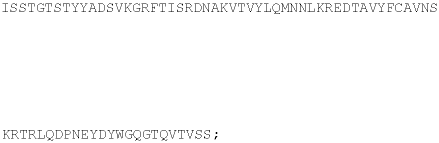
(SEQ ID NO: 26) 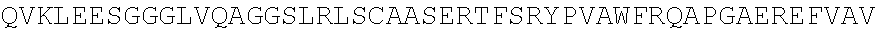 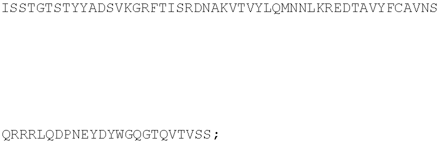
(SEQ ID NO: 27) 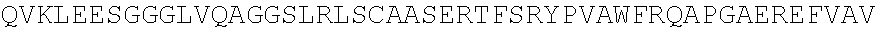 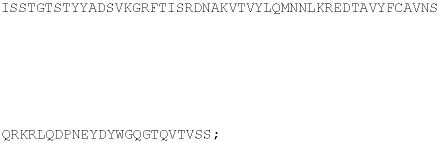
(SEQ ID NO: 28) 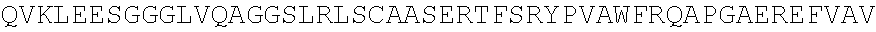 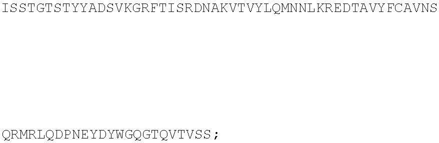
(SEQ ID NO: 29) 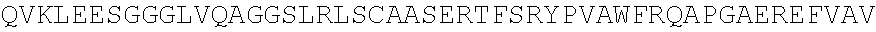 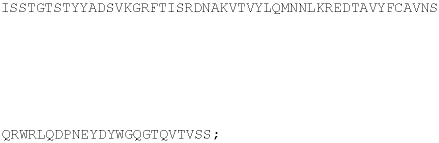
(SEQ ID NO: 30) 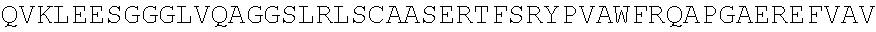 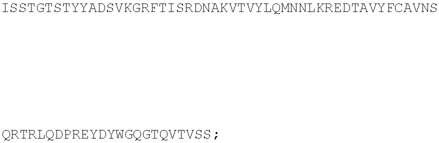
(SEQ ID NO: 31) 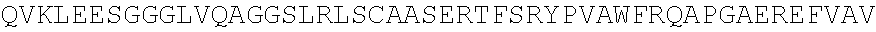 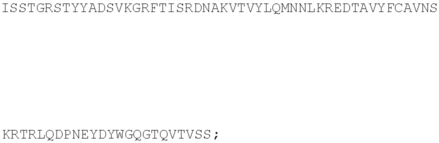
(SEQ ID NO: 32) 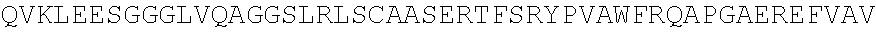 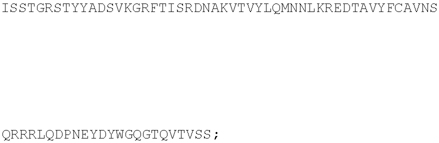
(SEQ ID NO: 33) 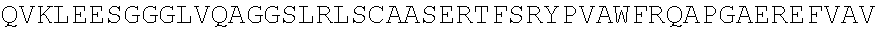 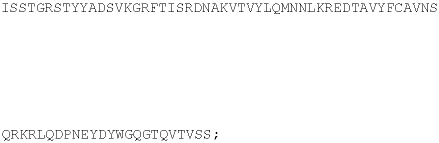
(SEQ ID NO: 34) 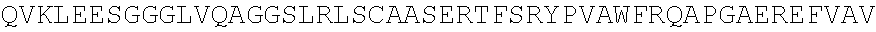 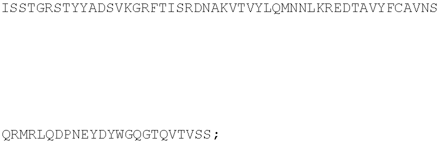
(SEQ ID NO: 35) 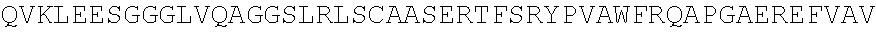 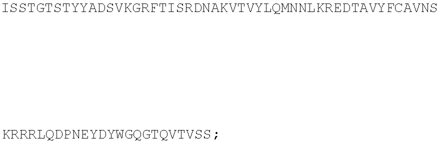
(SEQ ID NO: 36) 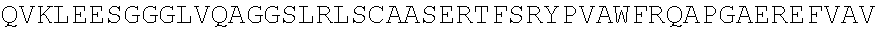 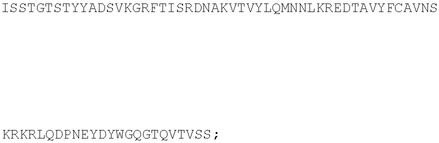
(SEQ ID NO: 37) 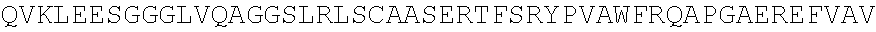 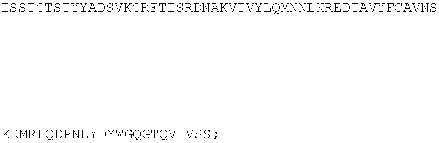
(SEQ ID NO: 38) 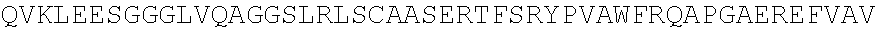 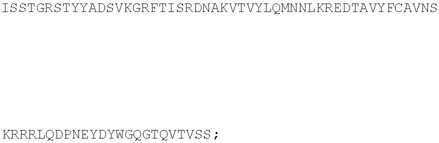
(SEQ ID NO: 39) 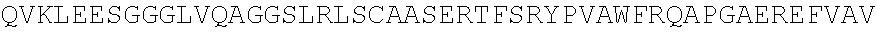 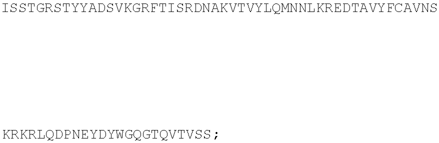
(SEQ ID NO: 40) 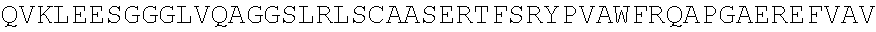 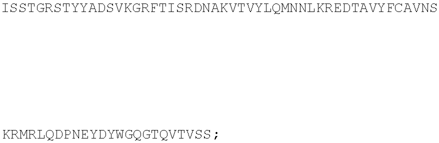
or a sequence substantially identical thereto.

6. The isolated, purified or recombinant antibody or fragment of one of claims 1, which neutralizes the cytotoxicity of TcdA with an EC50 in the nanomolar concentration range.

7. The isolated, purified or recombinant antibody or fragment selected from the group consisting of SEQ ID NO: 32 and SEQ ID NO: 38, which neutralizes the cytotoxicity of TcdA with an EC50 below the concentration of 100 nM and preferably with an EC50 of 20 nM or lower.

8. The isolated, purified or recombinant antibody or fragment of claim 1, wherein the antibody is a single-domain antibody (sdAb).

9. The isolated, purified or recombinant antibody or fragment thereof of claim 8, wherein the antibody or fragment thereof is in a multivalent display format.

10. A nucleic acid molecule encoding the isolated, purified or recombinant antibody or fragment thereof of claim 1.

11. A vector comprising the nucleic acid molecule of claim 10.

12. The isolated or purified antibody or fragment thereof of claim 1, wherein the antibody or fragment thereof is immobilized onto a surface.

13. The isolated or purified antibody or fragment thereof of claim 1, wherein the antibody or fragment thereof is linked to a cargo molecule.

14. The isolated or purified antibody or fragment thereof of claim 13, wherein the cargo molecule is a detectable agent, a therapeutic, a drug, a peptide, a carbohydrate moiety, an enzyme, or a cytotoxic agent; one or more liposomes loaded with a detectable agent, a therapeutic, a drug, a peptide, an enzyme, or a cytotoxic agent; or one or more nanoparticle, nanowire, nanotube, or quantum dots.

15. A composition comprising one or more than one isolated or purified antibody or fragment thereof of claim 1 and a pharmaceutically-acceptable carrier, diluent, or excipient.

16. A method of treating a Clostridium difficile infection, comprising administering the isolated or purified antibody or fragment thereof of claim 1 to a subject in need thereof.

17. A method of capturing Clostridium difficile toxins, comprising contacting a sample with one or more than one isolated or purified antibody or fragment thereof of claim 12, and allowing the toxin(s) to bind to the isolated or purified antibody or fragment thereof.

18. A method of detecting Clostridium difficile toxins, comprising contacting a sample with one or more than one isolated or purified antibody or fragment thereof of claim 13, and detecting the bound antibody or fragment thereof using a suitable detection and/or imaging technology.


SEQUENCE LISTING (40 sequences)

SEQ ID NO: 1 - protein, 8 aa, Artificial Sequence
  CDR1 (A26.8-generic or variant)
ERTFSRYP

SEQ ID NO: 2 - protein, 8 aa, Artificial Sequence
  CDR2 (A26.8-generic)
  misc_feature (4)..(4): is T or R
  misc_feature (6)..(6): is T, R or I
  misc_feature (8)..(8): is T or K
ISSXGXSX

SEQ ID NO: 3 - protein, 17 aa, Artificial Sequence
  CDR3 (A26.8-generic)
  misc_feature (5)..(5): is Q or K
  misc_feature (7)..(7): is T, R, K, M or W
  misc_feature (13)..(13): is N or R
AVNSXRXRLQDPXEYDY

SEQ ID NO: 4 - protein, 8 aa, Artificial Sequence
  CDR2 (A26.8 or variant)
ISSTGTST

SEQ ID NO: 5 - protein, 17 aa, Artificial Sequence
  CDR3 (A26.8 or variant)
AVNSQRTRLQDPNEYDY

SEQ ID NO: 6 - protein, 8 aa, Artificial Sequence
  CDR2 (A26.8 or variant)
ISSRGTST

SEQ ID NO: 7 - protein, 8 aa, Artificial Sequence
  CDR2 A26.8-T56R
ISSTGRST

SEQ ID NO: 8 - protein, 8 aa, Artificial Sequence
  CDR2 A26.8-T56I
ISSTGIST

SEQ ID NO: 9 - protein, 8 aa, Artificial Sequence
  CDR2 A26.8-T58K
ISSTGTSK

SEQ ID NO: 10 - protein, 17 aa, Artificial Sequence
  CDR3 A26.8 variant
AVNSKRTRLQDPNEYDY

SEQ ID NO: 11 - protein, 17 aa, Artificial Sequence
  CDR3 A26.8 variant
AVNSQRRRLQDPNEYDY

SEQ ID NO: 12 - protein, 17 aa, Artificial Sequence
  CDR3 A26.8 variant
AVNSQRKRLQDPNEYDY

SEQ ID NO: 13 - protein, 17 aa, Artificial Sequence
  CDR3 A26.8 variant
AVNSQRMRLQDPNEYDY

SEQ ID NO: 14 - protein, 17 aa, Artificial Sequence
  CDR3 A26.8-T103W
AVNSQRWRLQDPNEYDY

SEQ ID NO: 15 - protein, 17 aa, Artificial Sequence
  CDR3 A26.8-N109R
AVNSQRTRLQDPREYDY

SEQ ID NO: 16 - protein, 17 aa, Artificial Sequence
  CDR3 A26.8 variant
AVNSKRRRLQDPNEYDY

SEQ ID NO: 17 - protein, 17 aa, Artificial Sequence
  CDR3 A26.8 variant
AVNSKRKRLQDPNEYDY

SEQ ID NO: 18 - protein, 17 aa, Artificial Sequence
  CDR3 A26.8 variant
AVNSKRMRLQDPNEYDY

SEQ ID NO: 19 - protein, 124 aa, Artificial Sequence
  A26.8-generic
  misc_feature (54)..(54): is T or R
  misc_feature (56)..(56): is T, R or I
  misc_feature (58)..(58): is T or K
  misc_feature (101)..(101): is Q or K
  misc_feature (103)..(103): is T, R, K, M or W
  misc_feature (109)..(109): is N or R
QVKLEESGGGLVQAGGSLRLSCAASERTFSRYPVAWFRQAPGAEREFVAVISSXGXSXYYADSVKGRFTISRDNAKVTVYLQMNNLKREDTAVYFCAVNSXRXRLQDPXEYDYWGQGTQVTVSS

SEQ ID NO: 20 - protein, 124 aa, Artificial Sequence
  A26.8-wild-type
QVKLEESGGGLVQAGGSLRLSCAASERTFSRYPVAWFRQAPGAEREFVAVISSTGTSTYYADSVKGRFTISRDNAKVTVYLQMNNLKREDTAVYFCAVNSQRTRLQDPNEYDYWGQGTQVTVSS

SEQ ID NO: 21 - protein, 124 aa, Artificial Sequence
  A26.8-T54R
QVKLEESGGGLVQAGGSLRLSCAASERTFSRYPVAWFRQAPGAEREFVAVISSRGTSTYYADSVKGRFTISRDNAKVTVYLQMNNLKREDTAVYFCAVNSQRTRLQDPNEYDYWGQGTQVTVSS

SEQ ID NO: 22 - protein, 124 aa, Artificial Sequence [sequence omitted]

SEQ ID NO: 23 - protein, 124 aa, Artificial Sequence [sequence omitted]

SEQ ID NO: 24 - protein, 124 aa, Artificial Sequence
  A26.8-T58K
QVKLEESGGGLVQAGGSLRLSCAASERTFSRYPVAWFRQAPGAEREFVAVISSTGKSTYYADSVKGRFTISRDNAKVTVYLQMNNLKREDTAVYFCAVNSQRTRLQDPNEYDYWGQGTQVTVSS

SEQ ID NO: 25 - protein, 124 aa, Artificial Sequence
  A26.8-Q101K
QVKLEESGGGLVQAGGSLRLSCAASERTFSRYPVAWFRQAPGAEREFVAVISSTGTSTYYADSVKGRFTISRDNAKVTVYLQMNNLKREDTAVYFCAVNSKRTRLQDPNEYDYWGQGTQVTVSS

SEQ ID NO: 26 - protein, 124 aa, Artificial Sequence
  A26.8-T103R
QVKLEESGGGLVQAGGSLRLSCAASERTFSRYPVAWFRQAPGAEREFVAVISSTGTSTYYADSVKGRFTISRDNAKVTVYLQMNNLKREDTAVYFCAVNSQRRRLQDPNEYDYWGQGTQVTVSS

SEQ ID NO: 27 - protein, 124 aa, Artificial Sequence
  A26.8-T103K
QVKLEESGGGLVQAGGSLRLSCAASERTFSRYPVAWFRQAPGAEREFVAVISSTGTSTYYADSVKGRFTISRDNAKVTVYLQMNNLKREDTAVYFCAVNSQRKRLQDPNEYDYWGQGTQVTVSS

SEQ ID NO: 28 - protein, 124 aa, Artificial Sequence
  A26.8-T103M
QVKLEESGGGLVQAGGSLRLSCAASERTFSRYPVAWFRQAPGAEREFVAVISSTGTSTYYADSVKGRFTISRDNAKVTVYLQMNNLKREDTAVYFCAVNSQRMRLQDPNEYDYWGQGTQVTVSS

SEQ ID NO: 29 - protein, 124 aa, Artificial Sequence
  A26.8-T103W
QVKLEESGGGLVQAGGSLRLSCAASERTFSRYPVAWFRQAPGAEREFVAVISSTGTSTYYADSVKGRFTISRDNAKVTVYLQMNNLKREDTAVYFCAVNSQRWRLQDPNEYDYWGQGTQVTVSS

SEQ ID NO: 30 - protein, 124 aa, Artificial Sequence
  A26.8-N109R
QVKLEESGGGLVQAGGSLRLSCAASERTFSRYPVAWFRQAPGAEREFVAVISSTGTSTYYADSVKGRFTISRDNAKVTVYLQMNNLKREDTAVYFCAVNSQRTRLQDPREYDYWGQGTQVTVSS

SEQ ID NO: 31 - protein, 124 aa, Artificial Sequence
  A26.8-T56R,Q101K
QVKLEESGGGLVQAGGSLRLSCAASERTFSRYPVAWFRQAPGAEREFVAVISSTGRSTYYADSVKGRFTISRDNAKVTVYLQMNNLKREDTAVYFCAVNSKRTRLQDPNEYDYWGQGTQVTVSS

SEQ ID NO: 32 - protein, 124 aa, Artificial Sequence [sequence omitted]

SEQ ID NO: 33 - protein, 124 aa, Artificial Sequence [sequence omitted]

SEQ ID NO: 34 - protein, 124 aa, Artificial Sequence
  A26.8-T56R,T103M
QVKLEESGGGLVQAGGSLRLSCAASERTFSRYPVAWFRQAPGAEREFVAVISSTGRSTYYADSVKGRFTISRDNAKVTVYLQMNNLKREDTAVYFCAVNSQRMRLQDPNEYDYWGQGTQVTVSS

SEQ ID NO: 35 - protein, 124 aa, Artificial Sequence
  A26.8-Q101K,T103R
QVKLEESGGGLVQAGGSLRLSCAASERTFSRYPVAWFRQAPGAEREFVAVISSTGTSTYYADSVKGRFTISRDNAKVTVYLQMNNLKREDTAVYFCAVNSKRRRLQDPNEYDYWGQGTQVTVSS

SEQ ID NO: 36 - protein, 124 aa, Artificial Sequence
  A26.8-Q101K,T103K
QVKLEESGGGLVQAGGSLRLSCAASERTFSRYPVAWFRQAPGAEREFVAVISSTGTSTYYADSVKGRFTISRDNAKVTVYLQMNNLKREDTAVYFCAVNSKRKRLQDPNEYDYWGQGTQVTVSS

SEQ ID NO: 37 - protein, 124 aa, Artificial Sequence
  A26.8-Q101K,T103M
QVKLEESGGGLVQAGGSLRLSCAASERTFSRYPVAWFRQAPGAEREFVAVISSTGTSTYYADSVKGRFTISRDNAKVTVYLQMNNLKREDTAVYFCAVNSKRMRLQDPNEYDYWGQGTQVTVSS

SEQ ID NO: 38 - protein, 124 aa, Artificial Sequence
  A26.8-T56R,Q101K,T103R
QVKLEESGGGLVQAGGSLRLSCAASERTFSRYPVAWFRQAPGAEREFVAVISSTGRSTYYADSVKGRFTISRDNAKVTVYLQMNNLKREDTAVYFCAVNSKRRRLQDPNEYDYWGQGTQVTVSS

SEQ ID NO: 39 - protein, 124 aa, Artificial Sequence [sequence omitted]

SEQ ID NO: 40 - protein, 124 aa, Artificial Sequence [sequence omitted]